(19) United States
(12) Patent Application Publication
(10) Pub. No.: US 2023/0381718 A1
(43) Pub. Date: Nov. 30, 2023

(54) BARRIERS INCLUDING MOLECULES COVALENTLY BONDED TO AMPHIPHILIC MOLECULES, AND METHODS OF MAKING THE SAME

(71) Applicant: ILLUMINA CAMBRIDGE LIMITED, Cambridge (GB)

(72) Inventors: Antonio Conde-Gonzalez, Cambridge (GB); Alexandre Richez, Cambridge (GB); Davide Garoldini, Cambridge (GB); Oliver Uttley, Cambridge (GB); Charlotte Vacogne, Cambridge (GB)

(73) Assignee: ILLUMINA CAMBRIDGE LIMITED, Cambridge (GB)

(21) Appl. No.: 18/193,504

(22) Filed: Mar. 30, 2023

Related U.S. Application Data
(60) Provisional application No. 63/325,734, filed on Mar. 31, 2022.

(51) Int. Cl. B01D 69/12; B29C 39/14

(52) U.S. Cl. CPC B01D 69/122; B29C 39/14; B82Y 30/00

(57) ABSTRACT

Barriers including molecules covalently bonded to amphiphilic molecules, and methods of making the same, are provided herein. In some examples, a barrier between first and second fluids includes one or more layers comprising a plurality of amphiphilic molecules; and a first layer comprising a plurality of molecules covalently bonded to amphiphilic molecules of the plurality of amphiphilic molecules.

### CROSS-REFERENCE TO RELATED APPLICATIONS

This application claims the benefit of U.S. Provisional Patent Application No. 63/325,734, filed Mar. 31, 2022 and entitled “BARRIERS INCLUDING MOLECULES COVALENTLY BONDED TO AMPHIPHILIC MOLECULES, AND METHODS OF MAKING THE SAME”, the entire contents of which are incorporated by reference herein.

### FIELD

This application relates to barriers that include amphiphilic molecules.

### BACKGROUND

A significant amount of academic and corporate time and energy has been invested into using nanopores to sequence polynucleotides. For example, the dwell time has been measured for complexes of DNA with the Klenow fragment (KF) of DNA polymerase I atop a nanopore in an applied electric field. Or, for example, a current or flux-measuring sensor has been used in experiments involving DNA captured in an α-hemolysin nanopore. Or, for example, KF-DNA complexes have been distinguished on the basis of their properties when captured in an electric field atop an α-hemolysin nanopore. In still another example, polynucleotide sequencing is performed using a single polymerase enzyme complex including a polymerase enzyme and a template nucleic acid attached proximal to a nanopore, and nucleotide analogs in solution. The nucleotide analogs include charge blockade labels that are attached to the polyphosphate portion of the nucleotide analog such that the charge blockade labels are cleaved when the nucleotide analog is incorporated into a polynucleotide that is being synthesized. The charge blockade label is detected by the nanopore to determine the presence and identity of the incorporated nucleotide and thereby determine the sequence of a template polynucleotide. In still other examples, constructs include a transmembrane protein nanopore subunit and a nucleic acid handling enzyme.

However, such previously known devices, systems, and methods may not necessarily be sufficiently robust, reproducible, or sensitive and may not have sufficiently high throughput for practical implementation, e.g., demanding commercial applications such as genome sequencing in clinical and other settings that demand cost effective and highly accurate operation. Accordingly, what is needed are improved devices, systems, and methods for sequencing polynucleotides, which may include using membranes having nanopores disposed therein.

### SUMMARY

Barriers including molecules covalently bonded to amphiphilic molecules, and methods of making the same, are provided herein.

Some examples herein provide a barrier between first and second fluids. The barrier may include one or more layers comprising a plurality of amphiphilic molecules; and a first layer including a plurality of molecules covalently bonded to amphiphilic molecules of the plurality of amphiphilic molecules.

In some examples, the one or more layers include a first layer including a first plurality of amphiphilic molecules; a second layer including a second plurality of amphiphilic molecules contacting the first plurality of amphiphilic molecules. In some examples, the molecules of the first layer are covalently bonded to amphiphilic molecules of the first plurality of amphiphilic molecules.

In some examples, the first layer forms an outer surface of the barrier contacting the first fluid.

Some examples further include a second layer including a plurality of molecules covalently coupled to molecules of the first layer. In some examples, the second layer forms an outer surface of the barrier contacting the first fluid.

In some examples, at least some molecules of the first layer respectively couple to at least two molecules of the one or more layers comprising amphiphilic molecules.

In some examples, at least some molecules of the first layer couple to only one molecule of the one or more layers comprising amphiphilic molecules.

In some examples, hydrophilic groups of the one or more layers of amphiphilic molecules form an outer surface of the barrier contacting the second fluid.

In some examples, the amphiphilic molecules include molecules of a diblock copolymer, the molecules of the diblock copolymer respectively including a hydrophobic block coupled to a hydrophilic block. In some examples, the molecules of the first layer are coupled to the hydrophilic blocks of the diblock copolymer, and the hydrophobic blocks of the first and second pluralities of amphiphilic molecules contact one another within the barrier.

In some examples, the amphiphilic molecules include molecules of a triblock copolymer. In some examples, each molecule of the triblock copolymer includes first and second hydrophobic blocks and a hydrophilic block coupled to and between the first and second hydrophobic blocks. In some examples, the molecules of the first layer are coupled to the hydrophilic blocks of the triblock copolymer, and the hydrophobic blocks of the first and second pluralities of molecules contact one another within the barrier.

In some examples, each molecule of the triblock copolymer includes first and second hydrophilic blocks and a hydrophobic block coupled to and between the first and second hydrophilic blocks. In some examples, the molecules of the first layer are coupled to hydrophilic blocks of the triblock copolymer, and the hydrophobic blocks of the first and second pluralities of amphiphilic molecules contact one another within the barrier.

Some examples further include a nanopore within the barrier. In some examples, the nanopore includes α-hemolysin. In some examples, the nanopore includes MspA.

In some examples, the barrier is suspended by a barrier support defining an aperture, the one or more layers being suspended over the aperture.

Some examples herein provide a barrier between first and second fluids. The barrier may include a first layer including a first plurality of amphiphilic molecules, wherein the amphiphilic molecules of the first plurality of amphiphilic molecules include first reactive moieties.

In some examples, the first reactive moieties are selected from the group consisting of an amine group, a thiol group, a DBCO group, an azide group, an N-hydroxysuccinimide group, a biotin group, and a carboxyl group. In some examples, the first fluid includes reactive molecules including second reactive moieties selected to react with the first reactive moieties to form covalent bonds. In some examples, each of the reactive molecules includes at least two of the second reactive moieties.

In some examples, the barrier further includes a second layer including a second plurality of amphiphilic molecules contacting the first plurality of amphiphilic molecules. In some examples, hydrophilic groups of the second plurality of amphiphilic molecules form an outer surface of the barrier contacting the second fluid.

In some examples, the amphiphilic molecules include molecules of a diblock copolymer, the molecules of the diblock copolymer including a hydrophobic block coupled to a hydrophilic block. In some examples, the first reactive moieties are coupled to the hydrophilic blocks of the diblock copolymer, and the hydrophobic blocks of the first and second pluralities of amphiphilic molecules contact one another within the barrier.

In some examples, the amphiphilic molecules include molecules of a triblock copolymer. In some examples, each molecule of the triblock copolymer includes first and second hydrophobic blocks and a hydrophilic block coupled to and between the first and second hydrophobic blocks. In some examples, the first reactive moieties are coupled to the hydrophilic blocks of the triblock copolymer, and the hydrophobic blocks of the first and second pluralities of amphiphilic molecules contact one another within the barrier.

In some examples, the amphiphilic molecules include molecules of a triblock copolymer including first and second hydrophilic blocks having an approximate length A and a hydrophobic block having an approximate length B, the hydrophobic block being coupled to and between the first and second hydrophilic blocks. In some examples, the first reactive moieties are coupled to hydrophilic blocks of the triblock copolymer. The at least one layer may have a thickness of approximately A+2B.

In some examples, the barrier further includes a nanopore within the barrier. In some examples, the nanopore includes α-hemolysin. In some examples, the nanopore includes MspA.

In some examples, the barrier is suspended by a barrier support defining an aperture, the one or more layers being suspended across the aperture.

In some examples, the first fluid comprises activating molecules include a moiety selected to react with the first reactive moieties to form activated reactive moieties. In some examples, the first reactive moieties include a carboxyl group. In some examples, the first fluid includes reactive molecules including second reactive moieties selected to react with the activated reactive moieties to form covalent bonds. In some examples, each of the reactive molecules includes at least two of the second reactive moieties.

Some examples herein provide a method of forming a barrier between first and second fluids. The method may include forming one or more layers including a plurality of amphiphilic molecules, wherein the amphiphilic molecules include first reactive moieties; and using the first reactive moieties to covalently bond a first plurality of molecules to amphiphilic molecules of the plurality of amphiphilic molecules.

In some examples, the covalently bonded first plurality of molecules forms a first layer. In some examples, the covalently bonded first plurality of molecules forms an outer surface of the barrier contacting the first fluid.

In some examples, the first layer includes second reactive moieties. Some examples further include using the second reactive moieties to covalently bond a second plurality of molecules to the first layer. In some examples, the covalently bonded second plurality of molecules forms a second layer. In some examples, the second layer forms an outer surface of the barrier contacting the first fluid.

In some examples, at least some molecules of the first layer couple to at least two molecules of the at least one layer. In some examples, at least some molecules of the first layer couple to only one molecule of the at least one layer.

In some examples, forming the one or more layers includes forming a layer including a first plurality of the amphiphilic molecules, and forming another layer including a second plurality of the amphiphilic molecules contacting the first plurality of amphiphilic molecules.

In some examples, hydrophilic groups of the second plurality of amphiphilic molecules form an outer surface of the barrier contacting the second fluid.

In some examples, the amphiphilic molecules include molecules of a diblock copolymer, molecules of the diblock copolymer including a hydrophobic block coupled to a hydrophilic block. In some examples, the molecules of the first plurality of molecules are covalently coupled to the hydrophilic blocks of the diblock copolymer, and the hydrophobic blocks of the first and second pluralities of amphiphilic molecules contact one another within the barrier.

In some examples, the amphiphilic molecules include molecules of a triblock copolymer. In some examples, each molecule of the triblock copolymer includes first and second hydrophobic blocks and a hydrophilic block coupled to and between the first and second hydrophobic blocks. In some examples, the molecules of the first plurality of molecules are coupled to the hydrophilic blocks of the triblock copolymer, and the hydrophobic blocks of the first and second pluralities of amphiphilic molecules contact one another within the barrier.

In some examples, the amphiphilic molecules include molecules of a triblock copolymer including first and second hydrophilic blocks having an approximate length A and a hydrophobic block having an approximate length B, the hydrophobic block being coupled to and between the first and second hydrophilic blocks. In some examples, the first reactive moieties are coupled to hydrophilic blocks of the triblock copolymer. In some examples, the at least one layer has a thickness of approximately A+2B.

In some examples, the method further includes inserting a nanopore into the barrier. In some examples, the nanopore includes α-hemolysin. In some examples, the nanopore includes MspA.

In some examples, the first reactive moieties are selected from the group consisting of an amine group, a thiol group, a DBCO group, an azide group, an N-hydroxysuccinimide group, a biotin group, and a carboxyl group.

In some examples, the first fluid includes reactive molecules including second reactive moieties that react with the first reactive moieties to form the covalent bonds. In some examples, each of the reactive molecules including the second reactive moieties includes at least two of the second reactive moieties.

In some examples, using the first reactive moieties to covalently bond the first plurality of molecules to amphiphilic molecules of the first plurality of amphiphilic molecules includes: activating the first reactive moieties using an activating molecule; and covalently bonding the first plurality of molecules to the activated first reactive moieties. In some examples, the first reactive moieties include a carboxyl group. In some examples, the first fluid includes reactive molecules including second reactive moieties that react with the first reactive moieties to form the covalent bonds. In some examples, each of the reactive molecules includes at least two of the second reactive moieties.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIG. 1 schematically illustrates a cross-sectional view of an example nanopore composition and device including a barrier.

FIGS. 2A-2B schematically illustrate plan and cross-sectional views of further details of the nanopore composition and device of FIG. 1.

FIGS. 3A-3H schematically illustrate example operations for forming a barrier including molecules covalently bonded to amphiphilic molecules.

FIG. 4 schematically illustrates an alternative manner in which the operation described with reference to FIG. 3H may be performed.

FIG. 5 schematically illustrates an alternative barrier that may be used in operations such as described with reference to FIGS. 3A-3H.

FIG. 6 schematically illustrates another alternative barrier that may be used in operations such as described with reference to FIGS. 3A-3H.

FIGS. 7A-7C schematically illustrate further details of barriers using block copolymers which may be included in the nanopore composition and device of FIG. 1 and used in respective operations described with reference to FIGS. 3A-6.

FIG. 8 schematically illustrates use of a first set of example reactive moieties to couple molecules to a barrier.

FIG. 9 illustrates an example flow of operations in a method for forming a barrier including molecules covalently bonded to amphiphilic molecules.

FIG. 10 illustrates an experimental setup used to assess barrier stability improvement provided by reactions with molecules by evaluating barrier resistance to breakdown voltage.

FIG. 11 illustrates the voltage breakdown waveform used to assess polymeric barrier stability.

FIGS. 12A and 12B are plots of the measured barrier stability for barriers reacted with molecules under different conditions.

FIGS. 13A and 13B schematically illustrate example reaction products for the barriers reacted as described with reference to FIGS. 12A and 12B.

FIGS. 14A and 14B are plots of the measured barrier stability for the barriers of FIGS. 12A and 12B subsequently reacted with additional molecules.

FIG. 15 schematically illustrates example reaction products for the barriers reacted as described with reference to FIGS. 14A and 14B.

FIG. 16 schematically illustrates example reaction products for the barriers reacted as described with reference to FIG. 17.

FIG. 17 is a plot of the measured barrier stability for the barriers of FIG. 14A subsequently reacted with additional molecules.

FIG. 18 schematically illustrates a cross-sectional view of an example use of the composition and device of FIG. 1.

FIG. 19 schematically illustrates a cross-sectional view of another example use of the composition and device of FIG. 1.

FIG. 20 schematically illustrates a cross-sectional view of another example use of the composition and device of FIG. 1.

FIG. 21 schematically illustrates a cross-sectional view of another example use of the composition and device of FIG. 1.

FIG. 22 schematically illustrates use of a second set of example reactive moieties to couple molecules to a barrier.

FIG. 23 includes a plot of the measured stability of barriers reacted with molecules under different conditions, and an illustration of the voltage breakdown waveform used to assess barrier stability for this plot.

FIG. 24 is a plot of the measured stability of the barriers of FIG. 23, having nanopores inserted therein, and an illustration of the voltage breakdown waveform used to assess barrier stability for this plot.

FIG. 25 includes a plot of the measured stability of the barriers of FIG. 23, having nanopores inserted therein, and an illustration of the voltage breakdown waveform used to assess barrier stability for this plot.

FIG. 26 illustrates nonlimiting examples of block copolymers including various reactive moieties.

FIG. 27 schematically illustrates a cross-sectional view of another example use of the composition and device of FIG. 1.

### DETAILED DESCRIPTION

Barriers including molecules covalently bonded to amphiphilic molecules, and methods of making the same, are provided herein.

For example, nanopore sequencing may utilize a nanopore that is inserted into a barrier such as a polymeric membrane, and that includes an aperture through which ions and/or other molecules may flow from one side of the membrane to the other. Circuitry may be used to detect a sequence, e.g., of nucleotides. For example, during sequencing-by-synthesis (SBS), on a first side of the barrier, a polymerase adds the nucleotides to a growing polynucleotide in an order that is based on the sequence of a template polynucleotide to which the growing polynucleotide is hybridized. The sensitivity of the circuitry may be improved by using fluids with different compositions on respective sides of the barrier, for example to provide suitable ion transport for detection on one side of the barrier, while suitably promoting activity of the polymerase on the other side of the barrier. Accordingly, barrier stability is beneficial.

As provided herein, a barrier including amphiphilic molecules may be stabilized using layer-by-layer addition of molecules to the amphiphilic molecules. Illustratively, the amphiphilic molecules may be or include polymer chains that include functional groups at their respective hydrophilic ends. Molecules may be introduced to the fluid(s) in contact with the barrier, and the molecules may react with the functional groups in such a manner as to cross-link the amphiphilic molecules and/or provide additional functional groups with which other molecules similarly may be reacted in such a manner as to cross-link the amphiphilic molecules and/or provide additional functional groups with which still other molecules may be reacted in such a manner as to cross-link the amphiphilic molecules. Accordingly, the barrier may be expected to be sufficiently strong and stable for prolonged use under forces such as may be applied during use of a device including such a barrier, illustratively genomic sequencing.

First, some terms used herein will be briefly explained. Then, some example methods for forming barriers including molecules covalently bonded to amphiphilic molecules, and intermediate structures formed using such methods, will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or system, the term “comprising” means that the compound, composition, or system includes at least the recited features or components, but may also include additional features or components.

As used herein, the singular forms “a”, “an” and “the” include plural referents unless the content clearly dictates otherwise.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar, backbone, and/or phosphate moiety compared to naturally occurring nucleotides. Nucleotide analogues also may be referred to as “modified nucleic acids.” Example modified nucleobases include inosine, xanthine, hypoxanthine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates. Nucleotide analogues also include locked nucleic acids (LNA), peptide nucleic acids (PNA), and 5-hydroxylbutynl-2′-deoxyuridine (“super T”).

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof such as locked nucleic acids (LNA) and peptide nucleic acids (PNA). A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA, LNA, or PNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primer and a single stranded target polynucleotide, and can sequentially add nucleotides to the growing primer to form a “complementary copy” polynucleotide having a sequence that is complementary to that of the target polynucleotide. DNA polymerases may bind to the target polynucleotide and then move down the target polynucleotide sequentially adding nucleotides to the free hydroxyl group at the 3′ end of a growing polynucleotide strand. DNA polymerases may synthesize complementary DNA molecules from DNA templates. RNA polymerases may synthesize RNA molecules from DNA templates (transcription). Other RNA polymerases, such as reverse transcriptases, may synthesize cDNA molecules from RNA templates. Still other RNA polymerases may synthesize RNA molecules from RNA templates, such as RdRP. Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase.

Example DNA polymerases include Bst DNA polymerase, 9° Nm DNA polymerase, Phi29 DNA polymerase, DNA polymerase I (E. coli), DNA polymerase I (Large), (Klenow) fragment, Klenow fragment (3′-5′ exo-), T4 DNA polymerase, T7 DNA polymerase, Deep VentR™ (exo-) DNA polymerase, Deep VentR™ DNA polymerase, DyNAzyme™ EXT DNA, DyNAzyme™ II Hot Start DNA Polymerase, Phusion™ High-Fidelity DNA Polymerase, Therminator™ DNA Polymerase, Therminator™ II DNA Polymerase, VentR® DNA Polymerase, VentR® (exo-) DNA Polymerase, RepliPHI™ Phi29 DNA Polymerase, rBst DNA Polymerase, rBst DNA Polymerase (Large), Fragment (IsoTherm™ DNA Polymerase), MasterAmp™ AmpliTherm™, DNA Polymerase, Taq DNA polymerase, Tth DNA polymerase, Tfl DNA polymerase, Tgo DNA polymerase, SP6 DNA polymerase, Tbr DNA polymerase, DNA polymerase Beta, ThermoPhi DNA polymerase, and Isopol™ SD+ polymerase. In specific, nonlimiting examples, the polymerase is selected from a group consisting of Bst, Bsu, and Phi29. Some polymerases have an activity that degrades the strand behind them (3′ exonuclease activity). Some useful polymerases have been modified, either by mutation or otherwise, to reduce or eliminate 3′ and/or 5′ exonuclease activity.

Example RNA polymerases include RdRps (RNA dependent, RNA polymerases) that catalyze the synthesis of the RNA strand complementary to a given RNA template. Example RdRps include polioviral 3Dpol, vesicular stomatitis virus L, and hepatitis C virus NS5B protein. Example RNA Reverse Transcriptases. A non-limiting example list to include are reverse transcriptases derived from Avian Myelomatosis Virus (AMV), Murine Moloney Leukemia Virus (MMLV) and/or the Human Immunodeficiency Virus (HIV), telomerase reverse transcriptases such as (hTERT), SuperScript™ III, SuperScript™ IV Reverse Transcriptase, ProtoScript® II Reverse Transcriptase.

As used herein, the term “primer” is defined as a polynucleotide to which nucleotides may be added via a free 3′ OH group. A primer may include a 3′ block inhibiting polymerization until the block is removed. A primer may include a modification at the 5′ terminus to allow a coupling reaction or to couple the primer to another moiety. A primer may include one or more moieties, such as 8-oxo-G, which may be cleaved under suitable conditions, such as UV light, chemistry, enzyme, or the like. The primer length may be any suitable number of bases long and may include any suitable combination of natural and non-natural nucleotides. A target polynucleotide may include an “amplification adapter” or, more simply, an “adapter,” that hybridizes to (has a sequence that is complementary to) a primer, and may be amplified so as to generate a complementary copy polynucleotide by adding nucleotides to the free 3′ OH group of the primer.

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above example ranges. Accordingly, the definition of the term is intended to include all integer values greater than two.

As used herein, the term “double-stranded,” when used in reference to a polynucleotide, is intended to mean that all or substantially all of the nucleotides in the polynucleotide are hydrogen bonded to respective nucleotides in a complementary polynucleotide. A double-stranded polynucleotide also may be referred to as a “duplex.”

As used herein, the term “single-stranded,” when used in reference to a polynucleotide, means that essentially none of the nucleotides in the polynucleotide are hydrogen bonded to a respective nucleotide in a complementary polynucleotide.

As used herein, the term “target polynucleotide” is intended to mean a polynucleotide that is the object of an analysis or action, and may also be referred to using terms such as “library polynucleotide,” “template polynucleotide,” or “library template.” The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an amplification adapter that functions as a primer binding site, that flank(s) a target polynucleotide sequence that is to be analyzed. In particular examples, target polynucleotides may have different sequences than one another but may have first and second adapters that are the same as one another. The two adapters that may flank a particular target polynucleotide sequence may have the same sequence as one another, or complementary sequences to one another, or the two adapters may have different sequences. Thus, species in a plurality of target polynucleotides may include regions of known sequence that flank regions of unknown sequence that are to be evaluated by, for example, sequencing (e.g., SBS). In some examples, target polynucleotides carry an amplification adapter at a single end, and such adapter may be located at either the 3′ end or the 5′ end the target polynucleotide. Target polynucleotides may be used without any adapter, in which case a primer binding sequence may come directly from a sequence found in the target polynucleotide.

The terms “polynucleotide” and “oligonucleotide” are used interchangeably herein. The different terms are not intended to denote any particular difference in size, sequence, or other property unless specifically indicated otherwise. For clarity of description, the terms may be used to distinguish one species of polynucleotide from another when describing a particular method or composition that includes several polynucleotide species.

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, silica-based substrates can include silicon, silicon dioxide, silicon nitride, or silicone hydride. In some examples, substrates used in the present application include plastic materials or components such as polyethylene, polystyrene, poly(vinyl chloride), polypropylene, nylons, polyesters, polycarbonates, and poly(methyl methacrylate). Example plastics materials include poly(methyl methacrylate), polystyrene, and cyclic olefin polymer substrates. In some examples, the substrate is or includes a silica-based material or plastic material or a combination thereof. In particular examples, the substrate has at least one surface including glass or a silicon-based polymer. In some examples, the substrates can include a metal. In some such examples, the metal is gold. In some examples, the substrate has at least one surface including a metal oxide. In one example, the surface includes a tantalum oxide or tin oxide. Acrylamides, enones, or acrylates may also be utilized as a substrate material or component. Other substrate materials can include, but are not limited to gallium arsenide, indium phosphide, aluminum, ceramics, polyimide, quartz, resins, polymers and copolymers. In some examples, the substrate and/or the substrate surface can be, or include, quartz. In some other examples, the substrate and/or the substrate surface can be, or include, semiconductor, such as GaAs or ITO. The foregoing lists are intended to be illustrative of, but not limiting to the present application. Substrates can include a single material or a plurality of different materials. Substrates can be composites or laminates. In some examples, the substrate includes an organo-silicate material.

Substrates can be flat, round, spherical, rod-shaped, or any other suitable shape. Substrates may be rigid or flexible. In some examples, a substrate is a bead or a flow cell.

Substrates can be non-patterned, textured, or patterned on one or more surfaces of the substrate. In some examples, the substrate is patterned. Such patterns may include posts, pads, wells, ridges, channels, or other three-dimensional concave or convex structures. Patterns may be regular or irregular across the surface of the substrate. Patterns can be formed, for example, by nanoimprint lithography or by use of metal pads that form features on non-metallic surfaces, for example.

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that can be used in methods and compositions set forth herein include, but are not limited to, those commercially available from Illumina, Inc. (San Diego, CA).

As used herein, the term “electrode” is intended to mean a solid structure that conducts electricity. Electrodes may include any suitable electrically conductive material, such as gold, palladium, silver, or platinum, or combinations thereof. In some examples, an electrode may be disposed on a substrate. In some examples, an electrode may define a substrate.

As used herein, the term “nanopore” is intended to mean a structure that includes an aperture that permits molecules to cross therethrough from a first side of the nanopore to a second side of the nanopore, in which a portion of the aperture of a nanopore has a width of 100 nm or less, e.g., 10 nm or less, or 2 nm or less. The aperture extends through the first and second sides of the nanopore. Molecules that can cross through an aperture of a nanopore can include, for example, ions or water-soluble molecules such as amino acids or nucleotides. The nanopore can be disposed within a barrier, or can be provided through a substrate. Optionally, a portion of the aperture can be narrower than one or both of the first and second sides of the nanopore, in which case that portion of the aperture can be referred to as a “constriction.” Alternatively or additionally, the aperture of a nanopore, or the constriction of a nanopore (if present), or both, can be greater than 0.1 nm, 0.5 nm, 1 nm, 10 nm or more. A nanopore can include multiple constrictions, e.g., at least two, or three, or four, or five, or more than five constrictions. nanopores include biological nanopores, solid-state nanopores, or biological and solid-state hybrid nanopores.

Biological nanopores include, for example, polypeptide nanopores and polynucleotide nanopores. A “polypeptide nanopore” is intended to mean a nanopore that is made from one or more polypeptides. The one or more polypeptides can include a monomer, a homopolymer or a heteropolymer. Structures of polypeptide nanopores include, for example, an α-helix bundle nanopore and a β-barrel nanopore as well as all others well known in the art. Example polypeptide nanopores include aerolysin, α-hemolysin, Mycobacterium smegmatis porin A, gramicidin A, maltoporin, OmpF, OmpC, PhoE, Tsx, F-pilus, SP1, mitochondrial porin (VDAC), Tom40, outer membrane phospholipase A, CsgG, and Neisseria autotransporter lipoprotein (NaIP). Mycobacterium smegmatis porin A (MspA) is a membrane porin produced by Mycobacteria, allowing hydrophilic molecules to enter the bacterium. MspA forms a tightly interconnected octamer and transmembrane beta-barrel that resembles a goblet and includes a central constriction. For further details regarding α-hemolysin, see U.S. Pat. No. 6,015,714, the entire contents of which are incorporated by reference herein. For further details regarding SP1, see Wang et al., Chem. Commun., 49:1741-1743 (2013), the entire contents of which are incorporated by reference herein. For further details regarding MspA, see Butler et al., “Single-molecule DNA detection with an engineered MspA protein nanopore,” Proc. Natl. Acad. Sci. 105: 20647-20652 (2008) and Derrington et al., “Nanopore DNA sequencing with MspA,” Proc. Natl. Acad. Sci. USA, 107:16060-16065 (2010), the entire contents of both of which are incorporated by reference herein. Other nanopores include, for example, the MspA homolog from Nocardia farcinica, and lysenin. For further details regarding lysenin, see PCT Publication No. WO 2013/153359, the entire contents of which are incorporated by reference herein.

A “polynucleotide nanopore” is intended to mean a nanopore that is made from one or more nucleic acid polymers. A polynucleotide nanopore can include, for example, a polynucleotide origami.

A “solid-state nanopore” is intended to mean a nanopore that is made from one or more materials that are not of biological origin. A solid-state nanopore can be made of inorganic or organic materials. Solid-state nanopores include, for example, silicon nitride (SiN), silicon dioxide (SiO₂), silicon carbide (SiC), hafnium oxide (HfO₂), molybdenum disulfide (MoS₂), hexagonal boron nitride (h-BN), or graphene. A solid-state nanopore may comprise an aperture formed within a solid-state barrier, e.g., a barrier including any such material(s).

A “biological and solid-state hybrid nanopore” is intended to mean a hybrid nanopore that is made from materials of both biological and non-biological origins. Materials of biological origin are defined above and include, for example, polypeptides and polynucleotides. A biological and solid-state hybrid nanopore includes, for example, a polypeptide-solid-state hybrid nanopore and a polynucleotide-solid-state nanopore.

As used herein, a “barrier” or “membrane” is intended to mean a structure that normally inhibits passage of molecules from one side of the barrier to the other side of the barrier. The molecules for which passage is inhibited can include, for example, ions and water-soluble molecules such as nucleotides or amino acids. However, if a nanopore is disposed within a barrier, then the aperture of the nanopore may permit passage of molecules from one side of the barrier to the other side of the barrier. As one specific example, if a nanopore is disposed within a barrier, the aperture of the nanopore may permit passage of molecules from one side of the barrier to the other side of the barrier. Barriers include barriers of biological origin, such as lipid bilayers, and non-biological barriers such as solid-state barriers or substrates.

As used herein, “of biological origin” refers to material derived from or isolated from a biological environment such as an organism or cell, or a synthetically manufactured version of a biologically available structure.

As used herein, “solid-state” refers to material that is not of biological origin.

As used herein, “synthetic” refers to a barrier material that is not of biological origin (e.g., polymeric materials, synthetic phospholipids, solid-state barriers, or combinations thereof).

As used herein, a “polymeric barrier” refers to a synthetic barrier that primarily is composed of a polymer that is not of biological origin. In some examples, a polymeric barrier consists essentially of a polymer that is not of biological origin. A block copolymer is an example of a polymer that is not of biological origin and that may be included in the present barriers. A hydrophobic polymer with ionic end groups is another example of a polymer that is not of biological origin and that may be included in the present barriers. Because the present barriers relate to polymers that are not of biological origin, the terms “polymeric membrane,” “membrane,” “polymeric barrier,” and “barrier” may be used interchangeably when referring to the present barriers, even though the terms “barrier” and “membrane” generally may encompass other types of materials as well.

As used herein, the term “block copolymer” is intended to refer to a polymer having at least a first portion or “block” that includes a first type of monomer, and at least a second portion or “block” that is coupled directly or indirectly to the first portion and includes a second, different type of monomer. The first portion may include a polymer of the first type of monomer, or the second portion may include a polymer of the second type of monomer, or the first portion may include a polymer of the first type of monomer and the second portion may include a polymer of the second type of monomer. The first portion optionally may include an end group with a hydrophilicity that is different than that of the first type of monomer, or the second portion optionally may include an end group with a hydrophilicity that is different than that of the second type of monomer, or the first portion optionally may include an end group with a hydrophilicity that is different than that of the first type of monomer and the second portion optionally may include an end group with a hydrophilicity that is different than that of the second type of monomer. The end groups of any hydrophilic blocks may be located at an outer surface of a barrier formed using such hydrophilic blocks. Depending on the particular configuration, the end groups of any hydrophobic blocks may be located at an inner surface of the barrier or at an outer surface of a barrier formed using such hydrophobic blocks.

Block copolymers include, but are not limited to, diblock copolymers and triblock copolymers.

A “diblock copolymer” is intended to refer to a block copolymer that includes, or consists essentially of, first and second blocks coupled directly or indirectly to one another. The first block may be hydrophilic and the second block may be hydrophobic, in which case the diblock copolymer may be referred to as an “AB” copolymer where “A” refers to the hydrophilic block and “B” refers to the hydrophobic block.

A “triblock copolymer” is intended to refer to a block copolymer that includes, or consists essentially of, first, second, and third blocks coupled directly or indirectly to one another. The first and third blocks may include, or may consist essentially of, the same type of monomer as one another, and the second block may include a different type of monomer. In some examples, the first block may be hydrophobic, the second block may be hydrophilic, and the third block may be hydrophobic and includes the same type of monomer as the first block, in which case the triblock copolymer may be referred to as a “BAB” copolymer where “A” refers to the hydrophilic block and “B” refers to the hydrophobic blocks. In other examples, the first block may be hydrophilic, the second block may be hydrophobic, and the third block may be hydrophilic and includes the same type of monomer as the first block, in which case the triblock copolymer may be referred to as an “ABA” copolymer where “A” refers to the hydrophilic blocks and “B” refers to the hydrophobic block.

The particular arrangement of molecules of polymer chains (e.g., block copolymers) within a polymeric barrier may depend, among other things, on the respective block lengths, the type(s) of monomers used in the different blocks, the relative hydrophilicities and hydrophobicities of the blocks, the composition of the fluid(s) within which the barrier is formed, and/or the density of the polymeric chains within the barrier. During formation of the barrier, these and other factors generate forces between molecules of the polymeric chains which laterally position and reorient the molecules in such a manner as to substantially minimize the free energy of the barrier. The barrier may be considered to be substantially “stable” once the polymeric chains have completed these rearrangements, even though the molecules may retain some fluidity of movement within the barrier.

As used herein, the term “hydrophobic” is intended to mean tending to exclude water molecules. Hydrophobicity is a relative concept relating to the polarity difference of molecules relative to their environment. Non-polar (hydrophobic) molecules in a polar environment will tend to associate with one another in such a manner as to reduce contact with polar (hydrophilic) molecules to a minimum to lower the free energy of the system as a whole.

As used herein, the term “hydrophilic” is intended to mean tending to bond to water molecules. Polar (hydrophilic) molecules in a polar environment will tend to associate with one another in such a manner as to reduce contact with non-polar (hydrophobic) molecules to a minimum to lower the free energy of the system as a whole.

As used herein, the term “amphiphilic” is intended to mean having both hydrophilic and hydrophobic properties. For example, a block copolymer that includes a hydrophobic block and a hydrophilic block may be considered to be “amphiphilic.” Illustratively, AB copolymers, ABA copolymers, and BAB copolymers all may be considered to be amphiphilic.

As used herein, a “solution” is intended to refer to a homogeneous mixture including two or more substances. In such a mixture, a solute is a substance which is dissolved in another substance referred to as a solvent. A solution may include a single solute, or may include a plurality of solutes. An “aqueous solution” refers to a solution in which the solvent is, or includes, water.

A first liquid that forms a homogeneous mixture with a second liquid is referred to herein as being “miscible” or “soluble” with the second liquid.

As used herein, the term “electroporation” means the application of a voltage across a barrier such that a nanopore is inserted into the barrier.

As used herein, terms such as “covalently coupled” or “covalently bonded” refer to the forming of a chemical bond that is characterized by the sharing of pairs of electrons between atoms. For example, a covalently coupled molecule refers to a molecule that forms a chemical bond, as opposed to a non-covalent bond such as electrostatic interaction.

As used herein, “Cₐ to C_{b}” or “C_{a-b}” in which “a” and “b” are integers refer to the number of carbon atoms in the specified group. That is, the group can contain from “a” to “b”, inclusive, carbon atoms. Thus, for example, a “C₁ to C₄ alkyl” or “C₁₋₄ alkyl” or “C₁₋₄alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

The term “halogen” or “halo,” as used herein, means fluorine, chlorine, bromine, or iodine, with fluorine and chlorine being examples.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group may be designated as “C₁₋₄ alkyl” or similar designations. By way of example only, “C₁₋₄ alkyl” or “C₁₋₄alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 4 carbon atoms. The alkenyl group may be designated as “C₂₋₄ alkenyl” or similar designations. By way of example only, “C₂₋₄ alkenyl” indicates that there are two to four carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups include, but are in no way limited to, ethenyl, propenyl, butenyl, pentenyl, and hexenyl, and the like.

Groups that include an alkenyl group include optionally substituted alkenyl, cycloalkenyl, and heterocycloalkenyl groups.

As used herein, “alkynyl” refers to a straight or branched hydrocarbon chain containing one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 4 carbon atoms. The alkynyl group may be designated as “C₂₋₄ alkynyl” or similar designations. By way of example only, “C₂₋₄ alkynyl” or “C₂₋₄alkynyl” indicates that there are two to four carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like.

Groups that include an alkynyl group include optionally substituted alkynyl, cycloalkynyl, and heterocycloalkynyl groups.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some examples, the aryl group has 6 to 10 carbon atoms. The aryl group may be designated as “C₆₋₁₀ aryl,” “C₆ or C₁₀ aryl,” or similar designations. Examples of aryl groups include, but are not limited to, phenyl, naphthyl, azulenyl, and anthracenyl.

As used herein, “heterocycle” refers to a cyclic compound which includes atoms of carbon along with another atom (heteroatom), for example nitrogen, oxygen or sulfur. Heterocycles may be aromatic (heteroaryl) or aliphatic. An aliphatic heterocycle may be completely saturated or may contain one or more or two or more double bonds, for example the heterocycle may be a heterocycloalkyl. The heterocycle may include a single heterocyclic ring or multiple heterocyclic rings that are fused.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some examples, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

As used herein, “cycloalkyl” means a fully saturated carbocyclyl ring or ring system. Examples include cyclopropyl, cyclobutyl, cyclopentyl, and cyclohexyl.

As used herein, “cycloalkenyl” or “cycloalkene” means a carbocyclyl ring or ring system having at least one double bond, wherein no ring in the ring system is aromatic. An example is cyclohexenyl or cyclohexene. Another example is norbornene or norbornenyl.

As used herein, “heterocycloalkenyl” or “heterocycloalkene” means a carbocyclyl ring or ring system with at least one heteroatom in ring backbone, having at least one double bond, wherein no ring in the ring system is aromatic. In some examples, heterocycloalkenyl or heterocycloalkene ring or ring system is 3-membered, 4-membered, 5-membered, 6-membered, 7-membered, 8-membered, 9-membered, or 10-membered.

As used herein, “cycloalkynyl” or “cycloalkyne” means a carbocyclyl ring or ring system having at least one triple bond, wherein no ring in the ring system is aromatic. An example is cyclooctyne. Another example is bicyclononyne. Another example is dibenzocyclooctyne (DBCO).

As used herein, “heterocycloalkynyl” or “heterocycloalkyne” means a carbocyclyl ring or ring system with at least one heteroatom in ring backbone, having at least one triple bond, wherein no ring in the ring system is aromatic. In some examples, heterocycloalkynyl or heterocycloalkyne ring or ring system is 3-membered, 4-membered, 5-membered, 6-membered, 7-membered, 8-membered, 9-membered, or 10-membered.

As used herein, “heterocycloalkyl” means a non-aromatic cyclic ring or ring system containing at least one heteroatom in the ring backbone. Heterocycloalkyls may be joined together in a fused, bridged or spiro-connected fashion. Heterocycloalkyls may have any degree of saturation provided that at least one heterocyclic ring in the ring system is not aromatic. The heterocycloalkyl group may have 3 to 20 ring members (i.e., the number of atoms making up the ring backbone, including carbon atoms and heteroatoms), although the present definition also covers the occurrence of the term “heterocycloalkyl” where no numerical range is designated. The heterocycloalkyl group may also be a medium size heterocycloalkyl having 3 to 10 ring members. The heterocycloalkyl group could also be a heterocycloalkyl having 3 to 6 ring members. The heterocycloalkyl group may be designated as “3-6 membered heterocycloalkyl” or similar designations. In some six membered monocyclic heterocycloalkyls, the heteroatom(s) are selected from one up to three of O, N or S, and in some five membered monocyclic heterocycloalkyls, the heteroatom(s) are selected from one or two heteroatoms selected from 0, N, or S. Examples of heterocycloalkyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl (optionally substituted with halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

Where the compounds disclosed herein have at least one stereocenter, they may exist as individual enantiomers or diastereomers, or as mixtures of such isomers, including racemates. Separation of the individual isomers or selective synthesis of the individual isomers is accomplished by application of various methods which are well known to practitioners in the art. Where compounds disclosed herein are understood to exist in tautomeric forms, all tautomeric forms are included in the scope of the structures depicted. Unless otherwise indicated, all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

As used herein, the term “adduct” is intended to mean the product of a chemical reaction between two or more molecules, where the product contains all of the atoms of the molecules that were reacted.

As used herein, the term “linker” is intended to mean a molecule or molecules via which one element is attached to another element. For example, a linker may attach a first reactive moiety to a second reactive moiety. Linkers may be covalent, or may be non-covalent. Nonlimiting examples of covalent linkers include alkyl chains, polyethers, amides, esters, aryl groups, polyaryls, and the like. Nonlimiting examples of noncovalent linkers include host-guest complexation, cyclodextrin/norbornene, adamantane inclusion complexation with β-CD, DNA hybridization interactions, streptavidin/biotin, and the like.

As used herein, the term “activation” is intended to a reaction of a molecule that makes it easier to replace a moiety of the molecule with another moiety.

As used herein, the term “activating molecule” is intended to refer to any suitable chemical and/or enzymatic reagent that activates a moiety or molecule.

As used herein, the term “activated reactive moiety” is intended to refer to a reactive moiety that has been activated by an activating molecule.

As used herein, the term “activating a carboxyl group” is intended to refer to reacting the —OH group of the carboxyl group with any suitable chemical and/or enzymatic reagent(s) that make it easier to replace the —OH group with a nucleophilic moiety, such as an amine or hydroxyl group.

As used herein, the term “reactive molecule” is intended to refer to any suitable chemical and/or enzymatic reagent that includes a reactive moiety.

As used herein, the term “multifunctional molecule” is intended to refer to any suitable chemical and/or enzymatic reagent that includes at least two reactive moieties. In examples in which a multifunctional molecule includes exactly two reactive moieties, it may be referred to as “bifunctional.”

As used herein, the term “linker” is intended to mean a moiety, molecule, or molecules via which one element is attached to another element. Linkers may be covalent, or may be non-covalent. Nonlimiting examples of covalent linkers include moieties such as alkyl chains, polyethers, amides, esters, aryl groups, polyaryls, and the like. Nonlimiting examples of noncovalent linkers include host-guest complexation, cyclodextrin/norbornene, adamantane inclusion complexation with β-CD, DNA hybridization interactions, streptavidin/biotin, and the like.

As used herein, the terms “PEO”, “PEG”, “poly(ethylene oxide)”, and “poly(ethylene glycol)” are intended to be used interchangeably and refer to a polymer that comprises —[CH₂—CH₂—O]ₙ—. In some examples, n is between about 2 and about 100.

As used herein, the term “barrier support” is intended to refer to a structure that can suspend a barrier. A barrier support may define an aperture, such that a first portion of the barrier is suspended across the aperture, and a second portion of the barrier is disposed on, and supported by, the barrier. The barrier support may include any suitable arrangement of elements to define an aperture and suspend the barrier across the aperture. In some examples, a barrier support may include a substrate having an aperture defined therethrough, across which aperture the barrier may be suspended. Additionally, or alternatively, the barrier support may include one or more first features (such as one or more lips or ledges of a well within a substrate) that are raised relative to one or more second features (such as a bottom surface of the well), wherein a height difference between (a) the one or more first features and (b) the one or more second features defines an aperture across which a barrier may be suspended. The aperture may have any suitable shape, such as a circle, an oval, a polygon, or an irregular shape. The barrier support may include any suitable material or combination of materials. For example, the barrier support may be of biological origin, or may be solid state. Some examples, the barrier support may include, or may consist essentially of, an organic material, e.g., a curable resin such as SU-8; polytetrafluoroethylene (PTFE), poly(methyl methacrylate) (PMMA), parylene, or the like. Additionally, or alternatively, various examples, the barrier support may include, or may consist essentially of, an inorganic material, e.g., silicon nitride, silicon oxide, or molybdenum disulfide.

As used herein, the term “annulus” is intended to refer to a liquid that is adhered to a barrier support, located within a barrier, and extends partially into an aperture defined by the barrier support. As such, it will be understood that the annulus may follow the shape of the aperture of the barrier, e.g., may have the shape of a circle, an oval, a polygon, or an irregular shape.

Barriers Including Molecules Covalently Bonded to Amphiphilic Molecules, and Methods of Making the Same

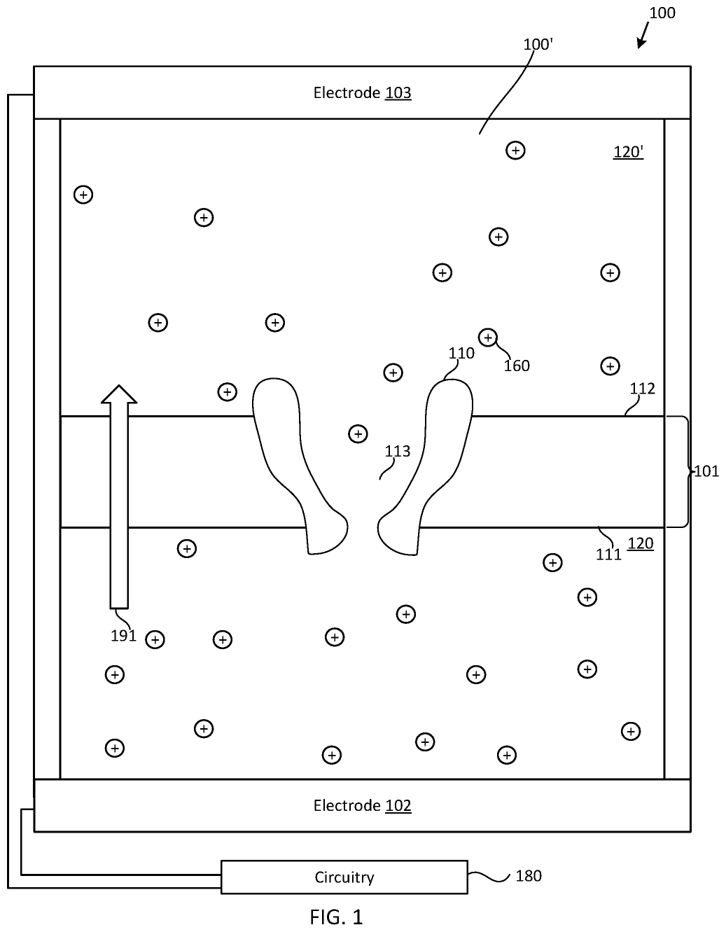

Barriers including molecules covalently bonded to amphiphilic molecules, and methods making the same, now will be described with reference to FIGS. 1-8 and 22.

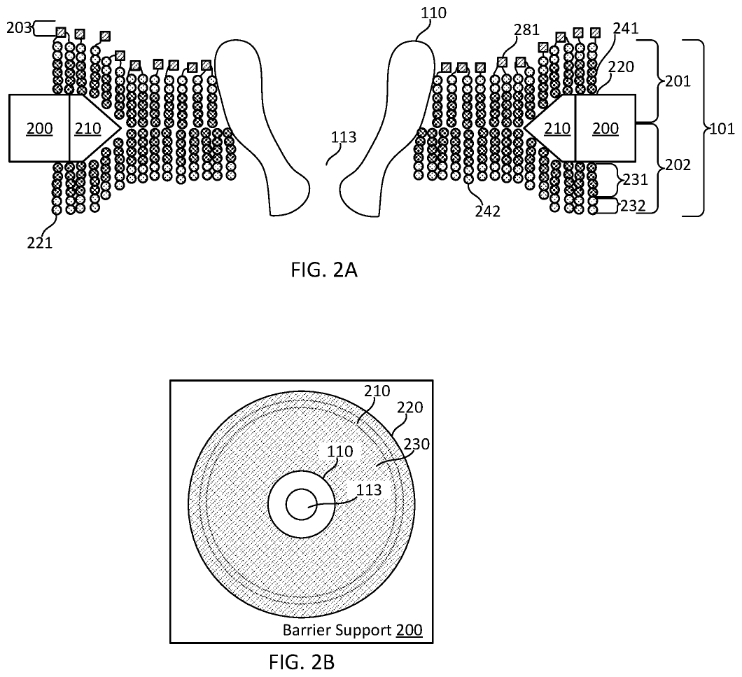

FIG. 1 schematically illustrates a cross-sectional view of an example nanopore composition and device 100 including a polymeric barrier. Device 100 includes fluidic well 100′ including barrier 101, such as a polymeric barrier, having first (trans) side 111 and second (cis) side 112, first fluid 120 within fluidic well 100′ and in contact with first side 111 of the barrier, and second fluid 120′ within the fluidic well and in contact with the second side 112 of the barrier. Barrier 101 may have any suitable structure that normally inhibits passage of molecules from one side of the barrier to the other side of the barrier, e.g., that normally inhibits contact between fluid 120 and fluid 120′. Illustratively, barrier 101 may include a polymeric barrier, which may include a diblock or triblock copolymer and may have a structure such as described in greater detail below with reference to FIG. 2A-2B, 3A-3H, 4, 5, 6, 7A-7C, or 22.

First fluid 120 may have a first composition including a first concentration of a salt 160, which salt may be represented for simplicity as positive ions although it will be appreciated that counterions also may be present. Second fluid 120′ may have a second composition including a second concentration of the salt 160 that may be the same as, or different, than the first concentration. Any suitable salt or salts 160 may be used in first and second fluids 120, 120′, e.g., ranging from common salts to ionic crystals, metal complexes, ionic liquids, or even water-soluble organic ions. For example, the salt may include any suitable combination of cations (such as, but not limited to, H, Li, Na, K, NH₄, Ag, Ca, Ba, and/or Mg) with any suitable combination of anions (such as, but not limited to, OH, Cl, Br, I, NO₃, ClO₄, F, SO₄, and/or CO₃²⁻). In one nonlimiting example, the salt includes potassium chloride (KCl). It will also be appreciated that the first and second fluids optionally may include any suitable combination of other solutes. Illustratively, first and second fluids 120, 120′ may include an aqueous buffer (such as N-(2-hydroxyethyl)piperazine-N′-2-ethanesulfonic acid (HEPES), commercially available from Fisher BioReagents).

Still referring to FIG. 1 device 100 further may include nanopore disposed within barrier 101 and providing aperture 113 fluidically coupling first side 111 to second side 112. As such, aperture 113 of nanopore 110 may provide a pathway for fluid 120 and/or fluid 120′ (e.g., salt 160) to flow through barrier 101. Nanopore 110 may include a solid-state nanopore, a biological nanopore (e.g., MspA such as illustrated in FIG. 1), or a biological and solid-state hybrid nanopore. Nonlimiting examples and properties of barriers and nanopores are described elsewhere herein, as well as in U.S. Pat. No. 9,708,655, the entire contents of which are incorporated by reference herein. In a manner such as illustrated in FIG. 1, device 100 optionally may include first electrode 102 in contact with first fluid 120, second electrode 103 in contact with second fluid 120′, and circuitry 180 in operable communication with the first and second electrodes and configured to detect changes in an electrical characteristic of the aperture. Such changes may, for example, be responsive to any suitable stimulus. Indeed, it will be appreciated that the present methods, compositions, and devices may be used in any suitable application or context, including any suitable method or device for sequencing, e.g., polynucleotide sequencing.

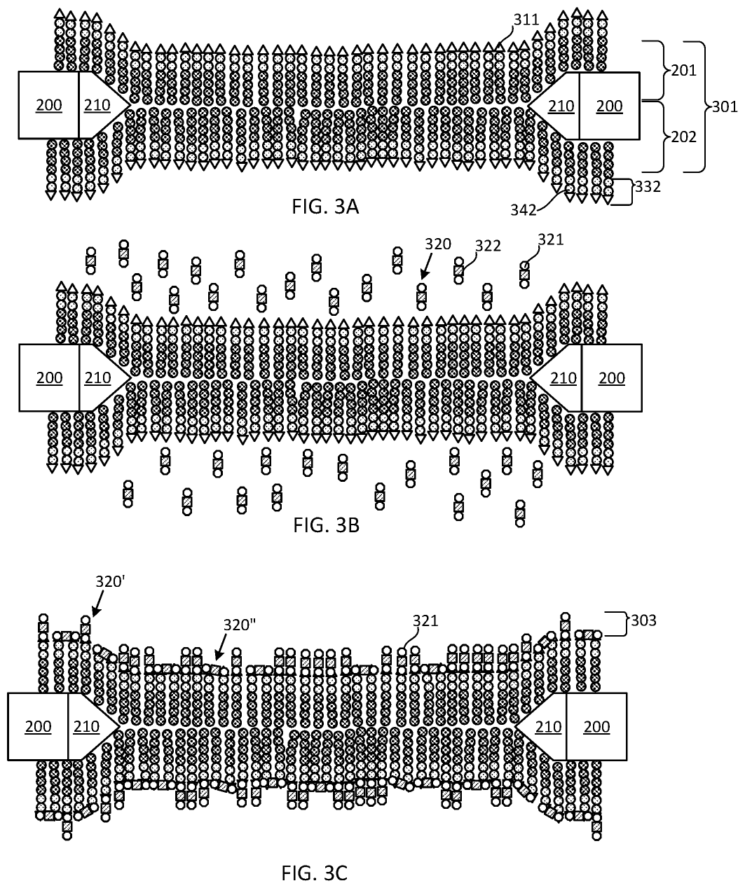

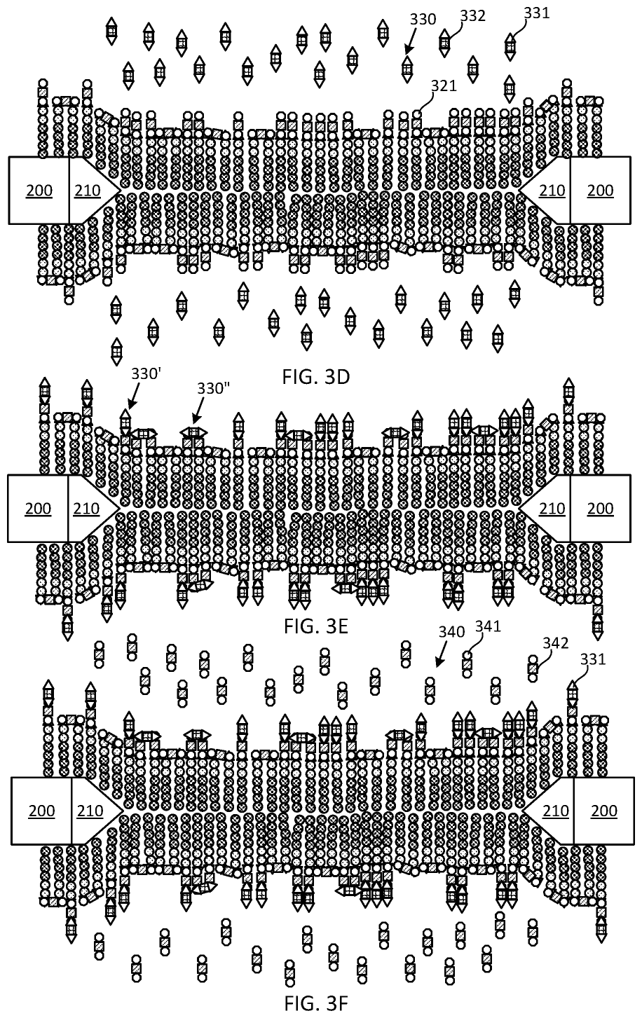

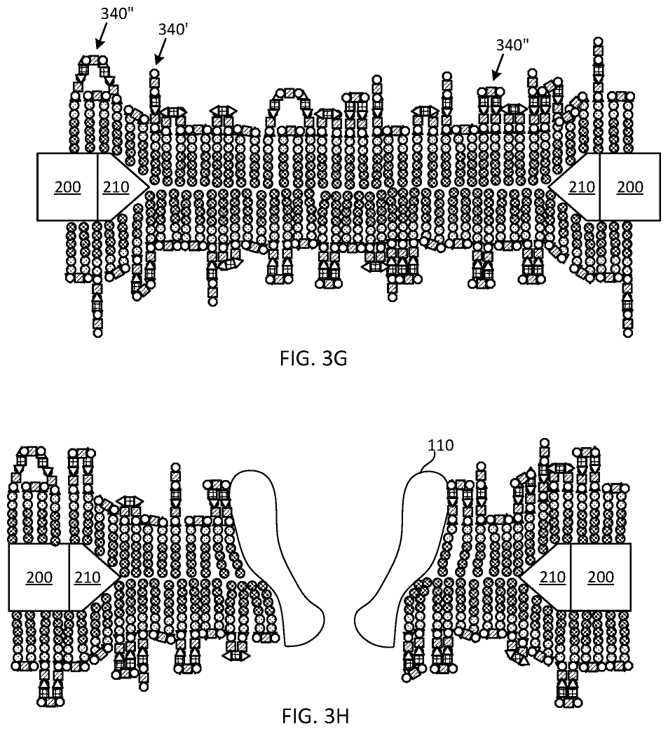

In some examples, polymeric barrier 101 between first and second fluids 120, 120′ includes a block copolymer. For example, FIGS. 2A-2B schematically illustrate plan and cross-sectional views of further details of the nanopore composition and device of FIG. 1. As illustrated in FIG. 2A, barrier 101 may include first layer 201 including a first plurality of amphiphilic molecules 221 and second layer 202 including a second plurality of the amphiphilic molecules contacting the first plurality of amphiphilic molecules. In the nonlimiting example illustrated in FIG. 2A, the copolymer is a diblock copolymer (AB), such that each molecule 221 includes a hydrophobic “B” block 231 (within which circles 241 with darker fill represent hydrophobic monomers) and a hydrophilic “A” block 232 (within which circles 242 with lighter fill represent hydrophilic monomers) coupled directly or indirectly thereto. In other examples such as will be described with reference to FIGS. 5 and 6, the copolymer instead may include a triblock copolymer (e.g., ABA or BAB, respectively). In the example illustrated in FIG. 2A, the hydrophilic blocks 232 of the first plurality of molecules 221 are coupled to a third plurality of molecules 281 that form a first outer surface of barrier 101, e.g., the surface of barrier 101 contacting fluid 120 on first side 111. The hydrophilic blocks 232 of the second plurality of molecules 221 may form a second outer surface of barrier 101, e.g., the surface of barrier 101 contacting fluid 120′ on second side 112. Alternatively, in a manner such as described with reference to FIGS. 3A-3H, the hydrophilic blocks 232 of the second plurality of molecules 221 optionally are coupled to a fourth plurality of the molecules 281 that form a second outer surface of barrier 101, e.g., the surface of barrier 101 contacting fluid 120′ on second side 112. The hydrophobic blocks 231 of the first and second pluralities of molecules 221 may contact one another within the barrier. In a manner such as illustrated in FIG. 2A and described in greater detail with reference to FIGS. 3A-3H, some of molecules 281 may be coupled to one of molecules 221 and/or some of molecules 281 may be coupled to two of molecules 221 and thus may be considered to cross-link molecules 221. As such, molecules 281 may strengthen and stabilize the barrier, resulting in improved performance and durability.

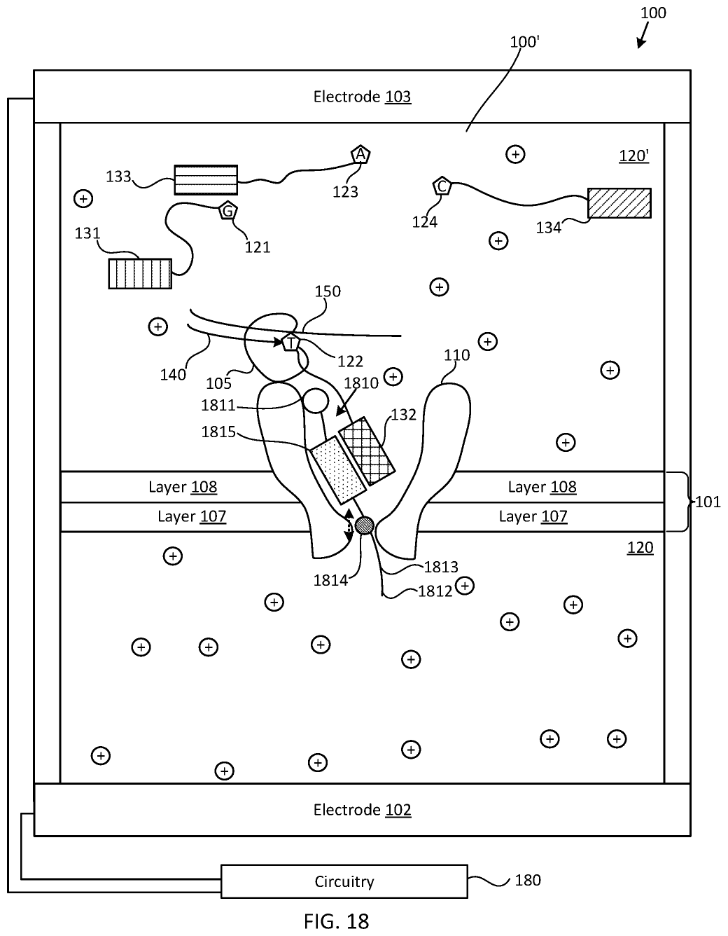

In the example illustrated in FIGS. 2A-2B, barrier 101 may be suspended using barrier support 200 defining aperture 230. For example, barrier support 200 may include a substrate having an aperture 230 defined therethrough, e.g., a substantially circular aperture. Additionally, or alternatively, the barrier support may include one or more features of a well in which the nanopore device is formed, such as a lip or ledge on either side of the well. Nonlimiting examples of materials which may be included in a barrier support are provided further above. An annulus 210 including hydrophobic (non-polar) solvent, and which also may include polymer chains and/or other compound(s), may adhere to barrier support 200 and may support a portion of barrier 101, e.g., may be located within barrier 101 (here, between layer 201 and layer 202). Additionally, annulus 210 may taper inwards in a manner such as illustrated in FIG. 2A. An outer portion of the molecules 221 of barrier 101 may be disposed on support 200 (e.g., the portion extending between aperture 230 and barrier periphery 220), while an inner portion of the molecules may form a freestanding portion of barrier 101 (e.g., the portion within aperture 210, a part of which is supported by annulus 210). Barrier 101 may be prepared, and nanopore 110 may be inserted into the freestanding portion of barrier 101, e.g., using operations such as now will be described with reference to FIGS. 3A-3H, 4, 5, 6, 7A-7C, 8, and 22. Although FIGS. 2A-2B illustrate nanopore 110 within barrier 101, it should be understood that the nanopore may be omitted, and that barrier 101 used for any suitable purpose. More generally, it should be appreciated that while the barriers described herein are particularly suitable for use with nanopores (e.g., for nanopore sequencing such as described with reference to FIGS. 18-21 and 27), the present barriers need not necessarily have nanopores inserted therein.

FIGS. 3A-3H schematically illustrate example operations for forming a barrier including molecules covalently bonded to amphiphilic molecules. FIG. 3A illustrates barrier 301. As illustrated in FIG. 3A, barrier 301 may be configured similarly as barrier 101 described as reference to FIGS. 2A-2B, e.g., may include layer 201 including a first plurality of amphiphilic molecules and layer 202 including a second plurality of amphiphilic molecules. The amphiphilic molecules of layer 201 (and optionally also of layer 202) may include first reactive moieties 311. In examples such as illustrated in FIG. 3A, the amphiphilic molecules include molecules of a diblock copolymer which are oriented such that the hydrophobic “B” sections of the AB diblock copolymer are oriented towards each other and disposed within the barrier, while the hydrophilic “A” sections form the outer surfaces of the barrier. As illustrated in FIG. 3A, hydrophilic “A” sections 332 may include reactive moieties 311, e.g., coupled to the terminal hydrophilic monomer 342. Suitable methods of forming suspended membranes, supported by a barrier support, are known in the art, such as “painting”, e.g., brush painting (manual), mechanical painting (e.g., using stirring bar), and bubble painting (e.g., using flow through the device).

Reactive moieties 311 may be reacted with molecules outside of barrier 301 in such a manner as to fully or partially cross-link the amphiphilic molecules with one another. For example, as illustrated in FIG. 3B, barrier 301 may be contacted with a fluid in which molecules 320 are dissolved. Molecules 320 may include second reactive moieties 321 and linker 322 coupled to second reactive moieties 321. In this example, molecules 320 are multifunctional (e.g., bifunctional), in that they each include at least two reactive moieties 321, each of which is available to react with one of first reactive moieties 311, and which are linked together by linker 322. Second reactive moieties 321 are selected so as to be chemically reactive with first moieties 311, e.g., so as to form products in which molecules 320 are covalently coupled to one or more of the amphiphilic molecules.

For example, FIG. 3C illustrates the products 320′, 320″ of reactions between molecules 320 and amphiphilic molecules 221. In this example, there is a mixture of products 320′ and 320″. For products 320′, one of second reactive moieties 321 of a molecule 320 reacted with one of first reactive moieties 311 while at least one other reactive moiety of that molecule did not react with a first reactive moiety and therefore remains available for further reaction such as will be described with reference to FIG. 3D. For products 320″, at least two of second reactive moieties 321 reacted with corresponding first reactive moieties 311 and therefore crosslink two molecules 221 and are not available for further reaction. It may be understood that controlling the relative proportion of products 320′ to 320″, e.g., through reaction conditions, may control the amount of cross-linking provided using reaction between molecules 320 and molecules 221. In some examples, products 320′ form substantially all of the products of the reactions, and accordingly there may be substantially no cross-linking between molecules 221 and substantially all of the products retain a second reactive moiety 321 that is available for further reaction. In other examples, products 320″ form substantially all of the products of the reactions, and accordingly there may be substantially complete cross-linking between molecules 221 and substantially none of the products retain a second reactive moiety 321 that is available for further reaction. In still other examples, the ratio of products 320′ to 320″ may be about 1:100 to about 100:1, e.g., about 1:50 to about 50:1, or about 1:20 to about 20:1, or about 1:10 to about 10:1. In any of such scenarios, the products 320′ and/or 320″ of such reactions may be considered to form a first layer 303 which is covalently coupled to amphiphilic molecules 221.

Any second reactive moieties 321 that are still available for reaction (e.g., second reactive moieties of products 320′) optionally may be reacted with further molecules outside of barrier 301 in such a manner as to still further fully or partially cross-link the amphiphilic molecules with one another. For example, as illustrated in FIG. 3D, barrier 301 including the products of reactions between the first and second reactive moieties 311, 321 may be contacted with a fluid in which molecules 330 are dissolved. Molecules 330 may include third reactive moieties 331 and linker 332 coupled to third reactive moieties 331. In this example, molecules 330 are multifunctional (e.g., bifunctional), in that they each include at least two reactive moieties 331, each of which is available to react with one of second reactive moieties 321, and which are linked together by linker 332. Third reactive moieties 331 are selected so as to be chemically reactive with second moieties 321, e.g., so as to form products in which molecules 330 are covalently coupled to one or more of molecules 320. In some examples, third reactive moieties 331 are the same type of reactive moiety as are first reactive moieties 211.

For example, FIG. 3E illustrates the products 330′, 330″ of reactions between molecules 330 and products 320′; note that products 320″ do not include available second reactive moieties 321 and therefore are unavailable to react with molecules 330. In this example, there is a mixture of products 330′ and 330″. For products 330′, one of third reactive moieties 331 of a molecule 330 reacted with one of second reactive moieties 321 while the other reactive moiet(ies) of that molecule did not react with a second reactive moiety and therefore remain available for further reaction such as will be described with reference to FIG. 3F. For products 330″, at least two of third reactive moieties 331 reacted with corresponding second reactive moieties 321 and therefore crosslink two products 320′ and are not available for further reaction. Accordingly, it may be understood that controlling the relative proportion of products 330′ to 330″, e.g., through reaction conditions, may control the amount of cross-linking provided using reaction between molecules 330 and product 320′. In some examples, products 330′ form substantially all of the products of the reactions, and accordingly there may be substantially no cross-linking between molecules 221 provided by products 330′ and substantially all of the products retain a third reactive moiety 331 that is available for further reaction. In other examples, products 330″ form substantially all of the products of the reactions, and accordingly there may be substantially complete cross-linking between molecules 221 provided by products 330″ and substantially none of the products retain a third reactive moiety 331 that is available for further reaction. In still other examples, the ratio of products 330′ to 330″ may be about 1:100 to about 100:1, e.g., about 1:50 to about 50:1, or about 1:20 to about 20:1, or about 1:10 to about 10:1. The products 330′, 330″ of such reactions may be considered to form a second layer 304 which is coupled to amphiphilic molecules 221 via first layer 303 to which layer 304 is covalently coupled.

Operations of adding additional layers of molecules that are covalently coupled to amphiphilic molecules 221, such as described with reference to FIGS. 3A-3E, may be repeated any suitable number of times. For example, any third reactive moieties 331 that are still available for reaction (e.g., third reactive moieties of products 330′) optionally may be reacted with still further molecules outside of barrier 301 in such a manner as to still further fully or partially cross-link the amphiphilic molecules with one another. For example, as illustrated in FIG. 3F, barrier 301 including the products of reactions between the second and third reactive moieties 321, 331 may be contacted with a fluid in which molecules 340 are dissolved. Molecules 340 may include fourth reactive moieties 341 and linker 342 coupled to fourth reactive moieties 341. In this example, molecules 340 are multifunctional (e.g., bifunctional), in that they each include at least two reactive moieties 341, each of which is available to react with one of third reactive moieties 331, and which are linked together by linker 342. Fourth reactive moieties 341 are selected so as to be chemically reactive with third moieties 331, e.g., so as to form products in which molecules 340 are covalently coupled to one or more of molecules 330. In some examples, fourth reactive moieties 341 are the same type of reactive moiety as reactive moieties 321.

For example, FIG. 3G illustrates the products 340′, 340″ of reactions between molecules 340 and products 330′; note that products 330″ do not include available third reactive moieties 331 and therefore are unavailable to react with molecules 340. In this example, there is a mixture of products 340′ and 340″. For products 340′, one of fourth reactive moieties 341 of a molecule 340 reacted with one of third reactive moieties 331 while the other reactive moiety of that molecule did not react with a second reactive moiety and therefore remains available for further reaction in a manner such as described herein. For products 340″, at least two of fourth reactive moieties 341 reacted with corresponding third reactive moieties 331 and therefore crosslink two products 330′ and are not available for further reaction. Accordingly, it may be understood that controlling the relative proportion of products 340′ to 340″, e.g., through reaction conditions, may control the amount of cross-linking provided using reaction between molecules 340 and product 330′. In some examples, products 340′ form substantially all of the products of the reactions, and accordingly there may be substantially no cross-linking between molecules 221 provided by products 340′ and substantially all of the products retain a fourth reactive moiety 341 that is available for further reaction. In other examples, products 340″ form substantially all of the products of the reactions, and accordingly there may be substantially complete cross-linking between molecules 221 provided by products 340″ and substantially none of the products retain a fourth reactive moiety 341 that is available for further reaction. In still other examples, the ratio of products 340′ to 340″ may be about 1:100 to about 100:1, e.g., about 1:50 to about 50:1, or about 1:20 to about 20:1, or about 1:10 to about 10:1. The products 340′, 340″ of any such reactions may be considered to form a third layer 305 which is covalently coupled to amphiphilic molecules 221 via first layer 303 and second layer 304.

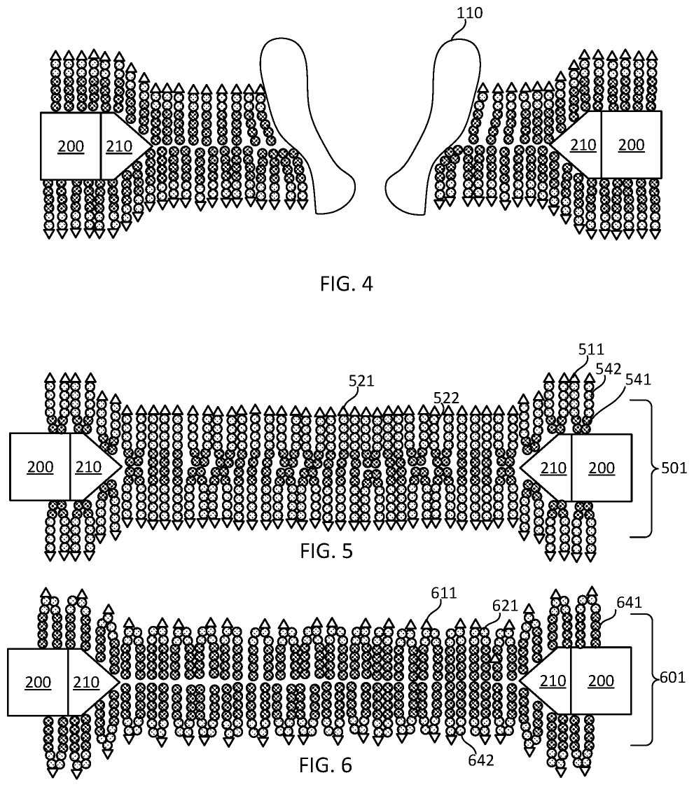

In some examples, following addition of any suitable number of layers of molecules to barrier 301, nanopore 110 may be inserted into the barrier in a manner such as illustrated in FIG. 3H. FIG. 4 schematically illustrates an alternative manner in which the operation described with reference to FIG. 3H may be performed. More specifically, as illustrated in FIG. 4, nanopore 110 may be inserted into suspended barrier 301 before addition of layer(s) to the barrier. The layer(s) then may be added in a manner such as described with reference to FIGS. 3A-3G. In still other examples, nanopore 110 may be inserted into the barrier during or between any suitable operations such as described with reference to FIGS. 3A-3G, e.g., between addition of the second and third layers. Nonlimiting examples of techniques for inserting nanopore 110 into the suspended barrier include electroporation, pipette pump cycle, and detergent assisted nanopore insertion. Tools for forming suspended barriers using synthetic polymers and inserting nanopores in the suspended barriers are commercially available, such as the Orbit 16 TC platform available from Nanion Technologies Inc. (California, USA).

Although FIGS. 3A-3H illustrate operations for adding molecules to amphiphilic molecules of a diblock copolymer, it will be appreciated that such operations similarly may be used to add molecules to other types of amphiphilic molecules, such as other types of polymers. FIG. 5 schematically illustrates an alternative barrier that may be used in operations such as described with reference to FIGS. 3A-3H. Barrier 501 illustrated in FIG. 5 may be suspended using barrier support 200 and annulus 20 in a manner such as described with reference to FIGS. 2A-2B. FIG. 5 illustrates barrier 501 including molecules of an ABA triblock copolymer including hydrophobic “B” sections 541 coupled to and between hydrophilic “A” sections 542. Each individual ABA molecule may be in one of two arrangements. For example, ABA molecules 521 may extend through the layer in a linear fashion, with an “A” section on each side of the barrier and the “B” section in the middle of the barrier. Or, for example, ABA molecules 522 may extend to the middle of the barrier and then fold back on themselves, so that both “A” sections are on the same side of the barrier and the “B” section is in the middle of the barrier. Accordingly, in this example, barrier 501 may be considered to be partially a single layer and partially a bilayer. In other examples (not specifically illustrated) in which barrier 501 substantially includes molecules 521 which extend through the barrier in linear fashion, barrier 501 may substantially be a monolayer. In still other examples (not specifically illustrated) in which barrier 501 substantially includes molecules 522 which extend to approximately the middle of the barrier and then fold back on themselves, barrier 501 may substantially be a bilayer.

First reactive moieties 511 may be coupled to hydrophilic sections 542, e.g., to the terminal hydrophilic monomer of such section. First reactive moieties 511 may be reacted with second reactive moieties of molecules 320 in a manner similar to that described with reference to FIGS. 3B-3C, and the products of such reactions then optionally may be reacted with additional molecule(s) in a manner similar to that described with reference to FIGS. 3D-3G. A nanopore optionally may be inserted into the barrier at any suitable time, e.g., before addition of the first layer, between addition of the first and second layers (if provided), between addition of the second and third layers (if provided), between addition of any subsequent layers (if provided), or after addition of the last layer.

FIG. 6 schematically illustrates another alternative barrier that may be used in operations such as described with reference to FIGS. 3A-3H. Barrier 601 illustrated in FIG. 6 may be suspended using barrier support 200 and annulus 20 in a manner such as described with reference to FIGS. 2A-2B. FIG. 6 illustrates barrier 601 including molecules of a BAB triblock copolymer including hydrophilic “A” sections 642 coupled to and between hydrophobic “B” sections 641. In this example, barrier 601 may have a bilayer architecture with the “B” sections 641 oriented towards each other. The hydrophobic ends of the BAB molecules 621 generally may located approximately in the middle of barrier 601, the molecules then extend towards either outer surface of the barriers, and then fold back on themselves. As such, both “B” sections are located in the middle of the barrier and the “A” section is on one side or the other of the barrier. First reactive moieties 611 may be coupled to hydrophilic sections 642, e.g., to one or more hydrophilic monomers of such section. First reactive moieties 611 may be reacted with second reactive moieties of molecules 320 in a manner similar to that described with reference to FIGS. 3B-3C, and the products of such reactions then optionally may be reacted with additional molecule(s) in a manner similar to that described with reference to FIGS. 3D-3G. A nanopore optionally may be inserted into the barrier at any suitable time, e.g., before addition of the first layer, between addition of the first and second layers (if provided), between addition of the second and third layers (if provided), between addition of any subsequent layers (if provided), or after addition of the last layer.

Although FIGS. 3A-3H, 4, 5, and 6 illustrate the presence of first reactive moieties 311, 511, or 611 on both sides of the barrier, it will be appreciated that such moieties may be reacted with molecules 320 on both sides of the barrier or alternatively may be reacted with molecules 320 on only one side of the barrier. For example, molecules 320 may be reacted with first reactive moieties 311, 511, 611 substantially only on the first side 111 of the barrier, e.g., by introducing molecules 320 substantially only to fluid on that side of the barrier. As such, the hydrophilic groups of on the second side 112 of the barrier may form an outer surface of the barrier contacting the second fluid. Or, for example, molecules 320 may be reacted with first reactive moieties 311, 511, 611 substantially only on the second side 112 of the barrier, e.g., by introducing molecules 320 substantially only to fluid on that side of the barrier. As such, the hydrophilic groups of on the first side 111 of the barrier may form an outer surface of the barrier contacting the second fluid. Or, for example, molecules 320 may be introduced to fluids on both sides of the barrier in a manner such as illustrated in FIG. 3B. Likewise, any other molecule(s) that it is desired to react with reactive moieties which are coupled to the barrier may be introduced to a fluid on any desired side or sides of the barrier.

It will further be appreciated that addition of molecules 330 and/or 340, and reaction of their respective reactive moieties on either or both sides of the barrier with reactive moieties that are coupled to the barrier, are optional. Illustratively, after the operations described with reference to FIGS. 3B and 3C, the barrier may include a first layer including a first plurality of amphiphilic molecules (e.g., AB, ABA, or BAB copolymer chains) a second layer including a second plurality of amphiphilic molecules (e.g., AB, ABA, or BAB copolymer chains) contacting the first plurality of amphiphilic molecules; and a third layer including a plurality of molecules covalently bonded to amphiphilic molecules of the first plurality of amphiphilic molecules (e.g., reaction products 320′ and/or 320″). As described with reference to FIG. 3C, in some examples at least some molecules of the third layer couple to two molecules of the first layer, and in these or other examples at least some molecules of the third layer couple to one molecule of the first layer. The third layer may be located on only one side of the barrier (e.g., on the first or second side 111, 112) or may be located on both sides of the barrier. If no further molecules 330 or 340 are reacted with reaction products 320′, then the third layer may form an outer surface of the barrier contacting fluid 120 or fluid 120′.

Alternatively, the operations described with reference to FIGS. 3D and 3E may be performed (e.g., to react molecules 330 with reaction products 320′), resulting in formation of a fourth layer including a plurality of molecules covalently coupled to molecules of the third layer (e.g., reaction products 330′ and/or 330″). If no further molecules 340 are reacted with reaction products 330′, then the fourth layer may form an outer surface of the barrier contacting fluid 120 or 120′. Alternatively, the operations described with reference to FIGS. 3F and 3G may be performed (e.g., to react molecules 340 with reaction products 330′), resulting in formation of a fifth layer including a plurality of molecules covalently coupled to molecules of the fourth layer (e.g., reaction products 340′ and/or 340″). If no further molecules are reacted with reaction products 340′, then the fourth layer may form an outer surface of the barrier contacting fluid 120 or 120′. If additional molecules are added, then their reaction products may form an outer surface of the barrier or may be further reacted with still further molecules.

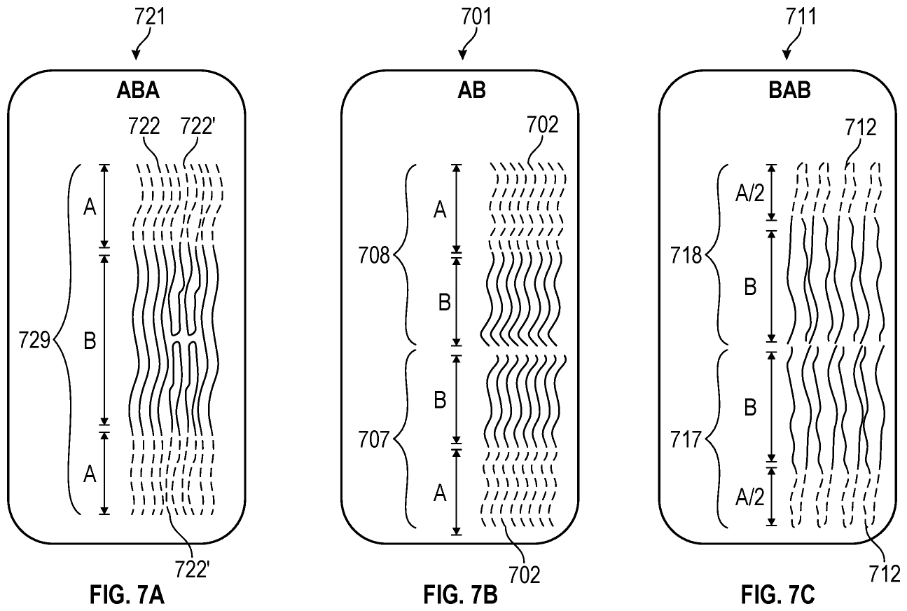

FIGS. 7A-7C schematically illustrate further details of barriers using block copolymers which may be included in the nanopore composition and device of FIG. 1 and used in respective operations described with reference to FIGS. 3A-6. It will be appreciated that such barriers suitably may be adapted for use in any other composition or device, and are not limited to use with nanopores. The hydrophilic blocks of the barriers described with reference to FIGS. 7A-7C may include first reactive moieties such as described elsewhere herein.

Referring now to FIG. 7A, barrier 721 uses a triblock “ABA” copolymer. Barrier 721 includes layer 729 which may contact both fluids 120 and 120′. Layer 729 includes a plurality of molecules 722 of a triblock ABA copolymer. As illustrated in FIG. 7A, each molecule 722 of the triblock copolymer includes first and second hydrophilic blocks, each denoted “A” and being approximately of length “A,” and a hydrophobic block disposed between the first and second hydrophilic blocks, denoted “B” and being approximately of length “B”. The hydrophilic A blocks at first ends of molecules 722 (the molecules forming layer 729) form a first outer surface of the barrier 721, e.g., contact fluid 120. The hydrophilic A blocks at second ends of molecules 722 form a second outer surface of the barrier 721, e.g., contact fluid 120′. The hydrophobic B blocks of the molecules 722 are within the barrier 711 in a manner such as illustrated in FIG. 7C. As illustrated, the majority of molecules 722 within layer 729 may extend substantially linearly and in the same orientation as one another. Optionally, as illustrated in FIG. 7A, some of the molecules 722′ may be folded at their B blocks, such that both of the hydrophilic A blocks of such molecules may contact the same fluid as one another. Accordingly, the example shown in FIG. 7A may be considered to be partially a single layer, and partially a bilayer. In other examples (not specifically illustrated), layer 729 may be entirely a single-layer or may be entirely a bilayer, e.g., as also described with reference to FIG. 1. Regardless of whether the membrane includes molecules 722 which extend substantially linearly and/or molecules 722′ which are folded, as illustrated in FIG. 7A, layer 729 may have a thickness of approximately 2A+B. In some examples, length A is about 1 RU to about 100 RU, or about 2 RU to about 100 RU e.g., about 10 RU to about 80 RU, or about 20 RU to about 50 RU, or about 50 RU to about RU. Additionally, or alternatively, in some examples, length B is about 5 RU to about 100 RU, e.g., about 10 RU to about 80 RU, or about 20 RU to about 50 RU, or about 50 RU to about RU. It will be appreciated that any end groups that are coupled to the hydrophilic or hydrophobic blocks contribute to the overall thickness of the barrier. Optionally, barrier 721 described with reference to FIG. 7A may be suspended across an aperture in a manner such as described with reference to FIG. 5.

Referring now to FIG. 7B, barrier 701 uses a diblock “AB” copolymer. Barrier 701 includes first layer 707 which may contact fluid 120 and second layer 708 which may contact fluid 120′ in a manner similar to that described with reference to FIG. 1. First layer 707 includes a first plurality of molecules 702 of a diblock AB copolymer, and second layer 708 includes a second plurality of the molecules 702 of the diblock AB copolymer. As illustrated in FIG. 7B, each molecule 702 of the diblock copolymer includes a hydrophobic block, denoted “B” and being approximately of length “B,” coupled to a hydrophilic block, denoted “A” and being approximately of length “A”. The hydrophilic A blocks of the first plurality of molecules 702 (the molecules forming layer 707) form a first outer surface of the barrier 701, e.g., contact fluid 120. The hydrophilic A blocks of the second plurality of molecules 702 (the molecules forming layer 708) form a second outer surface of the barrier 702, e.g., contact fluid 120′. The respective ends of the hydrophobic B blocks of the first and second pluralities of molecules contact one another within the barrier 701 in a manner such as illustrated in FIG. 7B. As illustrated, substantially all of the molecules 702 within layer 707 may extend substantially linearly and in the same orientation as one another, and similarly substantially all of the molecules 702 within layer 708 may extend substantially linearly and in the same orientation as one another (which is opposite that of the orientation the molecules within layer 707). Accordingly, first and second layers 707, 708 each may have a thickness of approximately A+B, and barrier 701 may have a thickness of approximately 2A+2B. In some examples, length A is about 1 repeating unit (RU) to about 100 RU, or about 2 RU to about 100 RU, or about 1 RU to about 50 RU, e.g., about 5 RU to about 40 RU, or about 10 RU to about 30 RU, or about 10 RU to about 20 RU, or about 20 RU to about 40 RU. Additionally, or alternatively, in some examples, length B is about 5 RU to about 100 RU, e.g., about 10 RU to about 80 RU, or about 20 RU to about 50 RU, or about 50 RU to about 80 RU. Optionally, barrier 701 described with reference to FIG. 7B may be suspended across an aperture in a manner such as described with reference to FIGS. 2A-2B.

Referring now to FIG. 7C, barrier 711 uses a triblock “BAB” copolymer. Barrier 711 includes first layer 717 which may contact fluid 120 and second layer 718 which may contact fluid 120′ in a manner similar to that described with reference to FIG. 1. First layer 717 includes a first plurality of molecules 712 of a triblock copolymer, and second layer 718 includes a second plurality of the molecules 712 of the triblock copolymer. As illustrated in FIG. 7C, each molecule 712 of the triblock copolymer includes first and second hydrophobic blocks, each denoted “B” and being approximately of length “B,” and a hydrophilic block disposed between the first and second hydrophobic blocks, denoted “A” and being approximately of length “A”. The hydrophilic A blocks of the first plurality of molecules 712 (the molecules forming layer 717) form a first outer surface of the barrier 711, e.g., contact fluid 120. The hydrophilic A blocks of the second plurality of molecules 712 (the molecules forming layer 718) form a second outer surface of the barrier 711, e.g., contact fluid 120′. The respective ends of the hydrophobic B blocks of the first and second pluralities of molecules contact one another within the barrier 711 in a manner such as illustrated in FIG. 7C. As illustrated, substantially all of the molecules 712 within layer 717 may extend in the same orientation as one another, and may be folded at the A block so that the A block can contact the fluid while the B blocks are interior to the barrier 711. Similarly, substantially all of the molecules 712 within layer 718 may extend in the same orientation as one another (which is opposite that of the orientation the molecules within layer 717), and may be folded at their A blocks so that the A blocks contact the fluid while the B blocks are interior to the barrier 711. Accordingly, first and second layers 717, 718 each may have a thickness of approximately A/2+B, and barrier 711 may have a thickness of approximately A+2B. In some examples, length A is about 2 RU to about 100 RU, e.g., about 10 RU to about 80 RU, or about 20 RU to about 50 RU, or about 50 RU to about 80 RU. Additionally, or alternatively, in some examples, length B is about 5 RU to about 100 RU, e.g., about 10 RU to about 80 RU, or about 20 RU to about 50 RU, or about 50 RU to about 80 RU. Optionally, barrier 711 described with reference to FIG. 7C may be suspended across an aperture in a manner such as described with reference to FIG. 6.

It will be appreciated that the layers of the various barriers provided herein may be configured so as to have any suitable dimensions. Illustratively, to form barriers of similar dimension as one another:

A-B-A triblock copolymer (FIG. 7A) may have 2 hydrophilic blocks, each of length A (each A block is of M_{w}=x) and 1 hydrophobic block of length B (M_{w} y); when self-assembled, those A-B-A triblock copolymers would form barriers with a top hydrophilic layer of length A, a core hydrophobic layer of length B, and a bottom hydrophilic layer of length A.

A-B diblock copolymer (FIG. 7B) may have 1 hydrophilic block of length A (M_{w}=x), and 1 hydrophobic block of length B (M_{w}=y/2); when self-assembled, those A-B diblock copolymers would form barriers with a top hydrophilic layer of length A, a core hydrophobic layer of length 2B, and a bottom hydrophilic layer of length A.

B-A-B triblock copolymer (FIG. 7C) may have 1 hydrophilic block of length A (M_{w}=x), and 2 hydrophobic blocks, each of length of B (each B block is of M_{w}=y/2); when self-assembled, those B-A-B triblock copolymers would form barriers with a top hydrophilic layer of length A/2, a core hydrophobic layer of length 2B, and a bottom hydrophilic layer of length A/2.

Additionally, or alternatively, the polymer packing into the layer(s) of the barrier may affect the hydrophilic ratio for each of the barriers, where hydrophilic ratio may be defined as the ratio between molecular mass of the hydrophilic block and the total molecular weight (M_{w}) of the block copolymer (BCP) (hydrophilic ratio=M_{w} hydrophilic block/M_{w} BCP). For example:
A-B-A triblock copolymer (FIG. 7A), hydrophilic ratio=x/(x+y);
A-B diblock copolymer (FIG. 7B), hydrophilic ratio=x/(x+y/2); and
B-A-B triblock copolymer (FIG. 7C), hydrophilic ratio=2x/(2x+y/2).

The present diblock and triblock copolymers may include any suitable combination of hydrophobic and hydrophilic blocks. In some examples, the hydrophilic A block may include a polymer selected from the group consisting of: N-vinyl pyrrolidone, polyacrylamide, zwitterionic polymer, hydrophilic polypeptide, nitrogen containing units, and poly(ethylene oxide) (PEO). Illustratively, the polyacrylamide may be selected from the group consisting of: poly(N-isopropyl acrylamide) (PNIPAM), and charged polyacrylamide, and phosphoric acid functionalized polyacrylamide. Nonlimiting examples of zwitterionic monomers that may be polymerized to form zwitterionic polymers include:

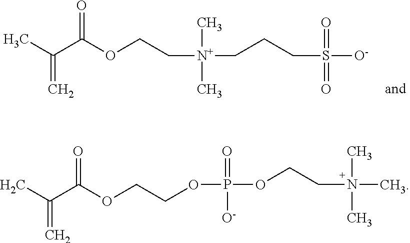

Nonlimiting examples of hydrophilic polypeptides include:

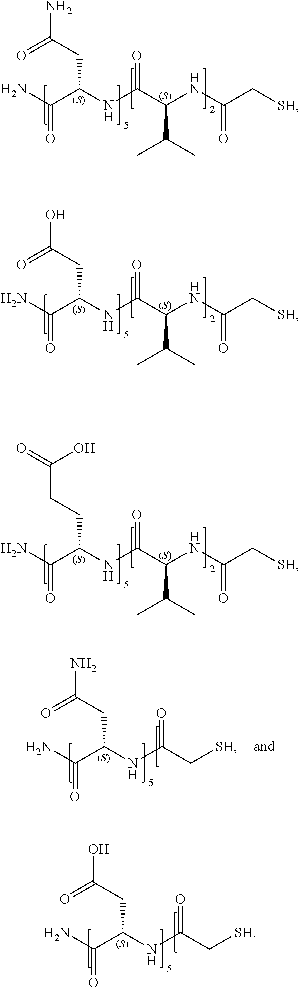

A nonlimiting example of a charged polyacrylamide is

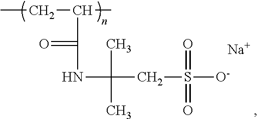

where n is between about 2 and about 100. Nonlimiting examples of nitrogen containing units include:

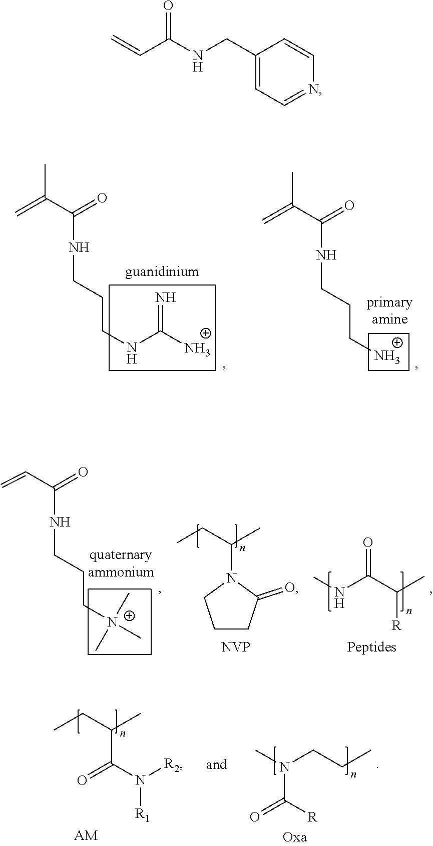

In some examples, the hydrophobic B block may include a polymer selected from the group consisting of: poly(dimethylsiloxane) (PDMS), polybutadiene (PBd), polyisoprene, polymyrcene, polychloroprene, hydrogenated polydiene, fluorinated polyethylene, polypeptide, and poly(isobutylene) (PIB). Nonlimiting examples of hydrogenated polydienes include saturated polybutadiene (PBu), saturated polyisoprene (PI), saturated poly(myrcene),

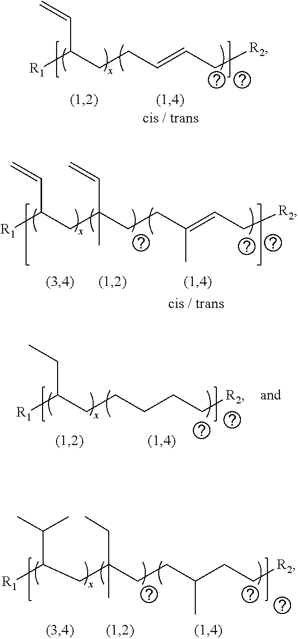

where n is between about 2 and about 100, x is between about 2 and about 100, y is between about 2 and about 100, z is between about 2 and about 100, R₁ is a functional group selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, any orthogonal functionality, and a hydrogen, and R₂ is a reactive moiety selected from the group consisting of a maleimide group, an allyl group, a propargyl group, a BCN group, a carboxylate group, an amine group, a thiol group, a DBCO group, an azide group, an N-hydroxysuccinimide group, a biotin group, a carboxyl group, an NHS-activated ester, and other activated esters. In other nonlimiting examples of hydrogenated polydienes, R₁ is a reactive moiety selected from the group consisting of a maleimide group, an allyl group, a propargyl group, a BCN group, a carboxylate group, an amine group, a thiol group, a DBCO group, an azide group, an N-hydroxysuccinimide group, a biotin group, a carboxyl group, an NHS-activated ester, and other activated esters. A nonlimiting example of fluorinated polyethylene is

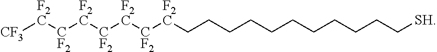 [SMILES: FC(F)(F)C(F)(F)C(F)(F)C(F)(F)C(F)(F)C(F)(F)C(F)(F)C(F)(F)CCCCCCCCCCCS]

Nonlimiting examples of hydrophobic polypeptides include (0<x<1):

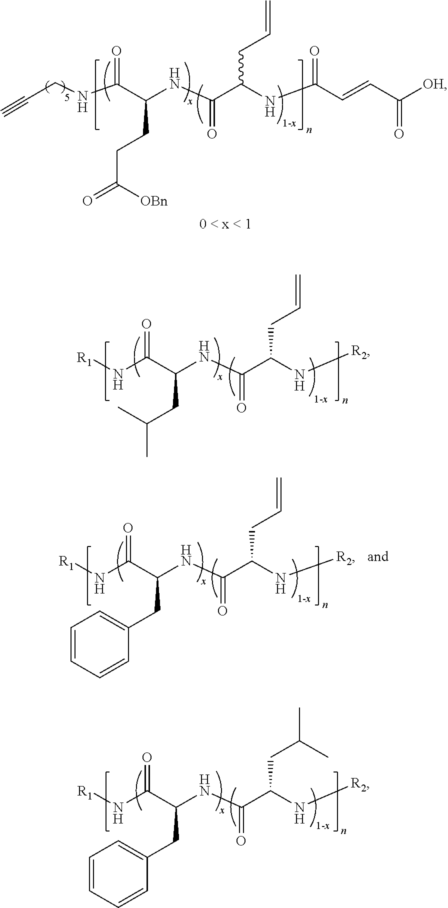

where n is between about 2 and about 100.

In one nonlimiting example, an AB diblock copolymer includes PDMS-b-PEO, where “-b-” denotes that the polymer is a block copolymer. In another nonlimiting example, an AB diblock copolymer includes PBd-b-PEO. In another nonlimiting example, an AB diblock copolymer includes PIB-b-PEO. In another nonlimiting example, a BAB triblock copolymer includes PDMS-b-PEO-b-PDMS. In another nonlimiting example, a BAB triblock copolymer includes PBd-b-PEO-b-PBd. In another nonlimiting example, a BAB triblock copolymer includes PIB-b-PEO-b-PIB. In another nonlimiting example, an ABA triblock copolymer includes PEO-b-PBd-b-PEO. In another nonlimiting example, an ABA triblock copolymer includes PEO-b-PDMS-b-PEO. In another nonlimiting example, an ABA triblock copolymer includes PEO-b-PIB-b-PEO. It will be appreciated that any suitable hydrophilic block(s) may be used with any suitable hydrophobic block(s). Additionally, in examples including two hydrophilic blocks, those blocks may be but need not necessarily include the same polymers as one another. Similarly, in examples including two hydrophobic blocks, those blocks may be but need not necessarily include the same polymers as one another.

The respective molecular weights, glass transition temperatures, and chemical structures of the hydrophobic and hydrophilic blocks suitably may be selected so as to provide the barrier with appropriate stability for use and ability to insert a nanopore. For example, the respective molecular weights of the hydrophobic and hydrophilic blocks may affect how thick each of the blocks (and thus layers of the barrier) are, and may influence stability as well as capacity to insert the nanopore, e.g., through electroporation, pipette pump cycle, or detergent assisted nanopore insertion. Additionally, or alternatively, the ratio of molecular weights of the hydrophilic and hydrophobic blocks may affect self-assembly of those blocks into the layers of the barrier. Additionally, or alternatively, the respective glass transition temperatures (T_{g}) of the hydrophobic and hydrophilic blocks may affect the lateral fluidity of the layers of the barrier; as such, in some examples it may be useful for the hydrophobic and/or hydrophilic blocks to have a T_{g} of less than the operating temperature of the device, e.g., less than room temperature, and in some examples less than about 0° C. Additionally, or alternatively, chemical structures of the hydrophobic and hydrophilic blocks may affect the way the chains get packed into the layers, and stability of those layers.

For nanopore sequencing applications, membrane fluidity can be considered beneficial. Without wishing to be bound by any theory, the fluidity of a block copolymer membrane is believed to be largely imparted by the physical property of the hydrophobic “B” blocks. More specifically, B blocks including “low T_{g}” hydrophobic polymers (e.g., having a T_{g} below around ° C.) may be used to generate membranes that are more fluid than those with B blocks including “high T_{g}” polymers (e.g., having a T_{g} above room temperature). For example, in certain examples, a hydrophobic B block of the copolymer has a T_{g} of less than about 20° C., less than about 0° C., or less than about −20° C.

Hydrophobic B blocks with a low T_{g} may be used to help maintain membrane flexibility under conditions suitable for performing nanopore sequencing, e.g., in a manner such as described with reference to FIG. 18, 19, 20, 21, or 27. In some examples, hydrophobic B blocks with a sufficiently low T_{g} for use in nanopore sequencing may include, or may consist essentially of, PIB, which may be expected to have a T_{g} in the range of about −75° C. to about −25° C. In other examples, hydrophobic B blocks with a sufficiently low T_{g} for use in nanopore sequencing may include, or may consist essentially of, PDMS, which may be expected to have a T_{g} in the range of about −135° C. (or lower) to about −115° C. In still other examples, hydrophobic B blocks with a sufficiently low T_{g} for use in nanopore sequencing may include, or may consist essentially of, PBd. Different forms of PBd may be used as B blocks in the present barriers. For example, the cis-1,4 form of PBd may be expected to have a T_{g} in the range of about −105° C. to about −85° C. Or, for example, the cis-1,2 form of PBd may be expected to have a T_{g} in the range of about −25° C. to about 0° C. Or, for example, the trans-1,4 form of PBd may be expected to have a T_{g} in the range of about −95° C. to about −5° C. In yet other examples, hydrophobic B blocks with a sufficiently low T_{g} for use in nanopore sequencing may include, or may consist essentially of, polymyrcene (PMyr), which may be expected to have a T_{g} in the range of about −75° C. to about −45° C. In yet other examples, hydrophobic B blocks with a sufficiently low T_{g} for use in nanopore sequencing may include, or may consist essentially of, polyisoprene (PIP). Different forms of PIP may be used as B blocks in the present barriers. For example, the cis-1,4 form of PIP may be expected to have a T_{g} in the range of about −85° C. to about −55° C. Or, for example, the trans-1,4 form of PIP may be expected to have a T_{g} in the range of about −75° C. to about −45° C.

Hydrophobic B blocks with a fully saturated carbon backbone, such as PIB, also may be expected to increase chemical stability of the block copolymer membrane. Additionally, or alternatively, branched structures within the hydrophobic B block, such as with PIB, may be expected to induce chain entanglement, which may be expected to enhance the stability of the block copolymer membrane. This may allow for a smaller hydrophobic block to be used, ameliorating the penalty of hydrophobic mismatch towards an inserted nanopore. Additionally, or alternatively, hydrophobic B blocks with relatively low polarity may be expected to be better electrical insulators, thus improving electrical performance of a device for nanopore sequencing (e.g., such as described with reference to FIG. 18-21 or 27).

In some examples of the AB copolymer shown below including PBd as the B block and PEO as the A block, R is a functional group selected from the group consisting of selected from the group consisting of amine group (—NH₂), thiol group (—SH), a dibenzocyclooctyne (DBCO) group, an azide group (—N₃), a biotin group, or a carboxyl group (—COOH); m=about 2 to about 100; and n=about 2 to about 100.

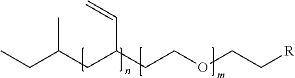

In some nonlimiting examples, R=—COOH; n=about 8 to about 50, m=about 1 to about 20. In some nonlimiting examples, R=—COOH; n=about 10 to about 15, m=about 5 to about

In some examples of the ABA copolymer shown below including one or more PIB blocks as the B block and PEO as the A block, R₁ and R₂ are independently moieties selected from the group consisting of amine group (—NH₂), thiol group (—SH), a dibenzocyclooctyne (DBCO) group, an azide group (—N₃), a biotin group, or a carboxyl group (—COOH); V is an optional group that corresponds to a bis-functional initiator from which the isobutylene may be propagated and can be tert-butylbenzene, a phenyl connected to the hydrophobic blocks via the para, meta, or ortho positions, naphthalene, another aromatic group, an alkane chain with between about 2 and about carbons, or another aliphatic group; m=about 2 to about 100; and n=about 2 to about 100. V may optionally be flanked by functional groups selected from the group consisting of a carboxylic acid, a carboxyl group, a methyl group, a hydroxyl group, a primary amine, a secondary amine, a tertiary amine, a biotin, a thiol, an azide, a propargyl group, an allyl group, an acrylate group, a zwitterionic group, a sulfate, a sulfonate, an alkyl group, an aryl group, any orthogonal functionality, and a hydrogen. When V is absent, only one PIB block is present and n=about 2 to about 100. L₁ and L₂ are independently linkers, which may include at least one moiety selected from the group consisting of an amide, a thioether (sulfide), a succinic group, a maleic group, an alkyl (such as methylene), an ether, and a product of a click reaction.

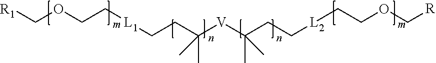

In some nonlimiting examples of the above structure, n=about 2 to about 50, and m=about 1 to about 50, R₁=R₂=COOH, V=tert-butylbenzene, and L₁=L₂=ethyl sulfide. In other nonlimiting examples, n=about 5 to about 20, m=about 2 to about 15, R₁=R₂=COOH, V=tert-butylbenzene, and L₁=L₂=ethyl sulfide. In other nonlimiting examples, n=about 13 to about 19, m=about 2 to about 5, R₁=R₂=COOH, V=tert-butylbenzene, and L₁=L₂=ethyl sulfide. In other nonlimiting examples, n=about 7 to about 13, m=about 7 to about 13, R₁=R₂=COOH, V=tert-butylbenzene, and L₁=L₂=ethyl sulfide. In particular, in one nonlimiting example (the structure of which is shown below), n=16, m=3, R₁=R₂=COOH, V=tert-butylbenzene, and L₁=L₂=ethyl sulfide. In another nonlimiting example (the structure of which is shown below, n=10, m=10, and R₁=R₂=COOH, V=tert-butylbenzene, and L₁=L₂=ethyl sulfide. In another nonlimiting example (the structure of which is shown below), n=16, m=8, R₁=R₂=methyl, V=tert-butylbenzene, and L₁=L₂=ethyl sulfide.

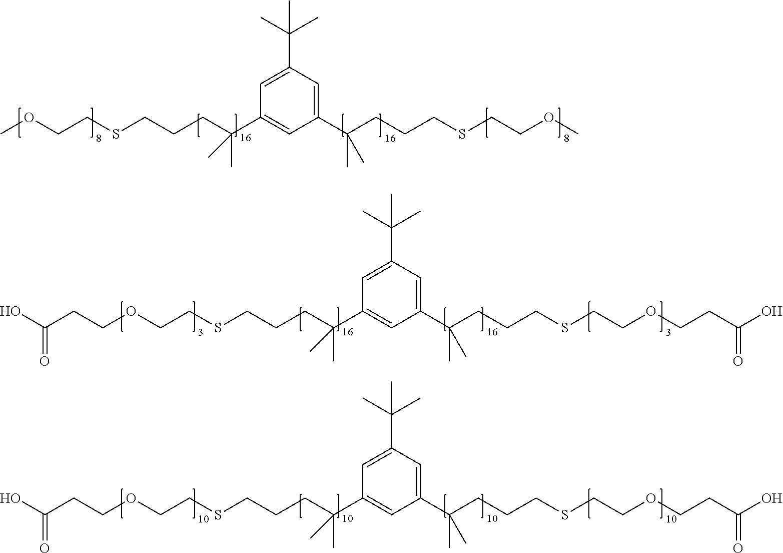

In some examples, multifunctional precursors may be sourced and used as precursors to the synthesis of bifunctional initiators to which V corresponds in the example further above. For example, the multifunctional precursor may be 5-tert-butylisophthalic acid (TBIPA) which can be synthesized into 1-(tert-butyl)-3,5-bis(2-methoxypropan-2-yl)benzene (TBDMPB) using reactions known in the art. In another example, TBIPA may be synthesized into 1-tert-butyl-3,5-bis(2-chloropropan-2-yl)benzene using reactions known in the art. The use of such bifunctional initiators allows cationic polymerization on both sides of the initiator, generating bifunctional PIBs, such as allyl-PIB-allyl, which can then be coupled to hydrophilic A blocks to generate ABA block copolymers including PIB as the B block. Here, although the bifunctional initiator may be located between first and second PIB polymers, it should be understood that the first and second PIB polymers and the bifunctional initiator (V) together may be considered to form a B block, e.g., of an ABA triblock copolymer.

In another nonlimiting example, an ABA triblock copolymer includes

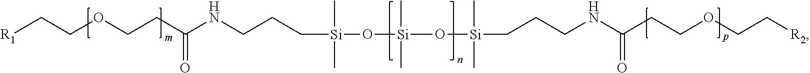

where m=about 2 to about 100, n=about 2 to about 100, p=about 2 to about 100, R₁ and R₂ are independently functional groups selected from the group consisting of amine group (—NH₂), thiol group (—SH), a dibenzocyclooctyne (DBCO) group, an azide group (—N₃), a biotin group, or a carboxyl group (—COOH). In some nonlimiting examples, m=about 2 to about 30, n=about 25 to about 45, p=about 2 to about 30, R₁ and R₂ are independently functional groups selected from the group consisting of amine group (—NH₂), thiol group (—SH), a dibenzocyclooctyne (DBCO) group, an azide group (—N₃), a biotin group, or a carboxyl group (—COOH). In some nonlimiting examples, m=about 2 to about 15, n=about 30 to about 40, p=about 2 to about 15, R₁ and R₂ are independently functional groups selected from the group consisting of amine group (—NH₂), thiol group (—SH), a dibenzocyclooctyne (DBCO) group, an azide group (—N₃), a biotin group, or a carboxyl group (—COOH). In some nonlimiting examples, m=about 7 to about 11, n=about 35 to about 40, p=about 7 to about 11, R₁ and R₂ are independently functional groups selected from the group consisting of amine group (—NH₂), thiol group (—SH), a dibenzocyclooctyne (DBCO) group, an azide group (—N₃), a biotin group, or a carboxyl group (—COOH). In some nonlimiting examples, m=about 2 to about 5, n=about 30 to about 37, p=about 2 to about 5, R₁ and R₂ are independently functional groups selected from the group consisting of amine group (—NH₂), thiol group (—SH), a dibenzocyclooctyne (DBCO) group, an azide group (—N₃), a biotin group, or a carboxyl group (—COOH).

In particular, as shown below, in one nonlimiting example, m=3, n=34, p=3, and R₁=R₂=COOH. In another nonlimiting example shown below, m=9, n=37, p=9, and R₁=R₂=COOH.

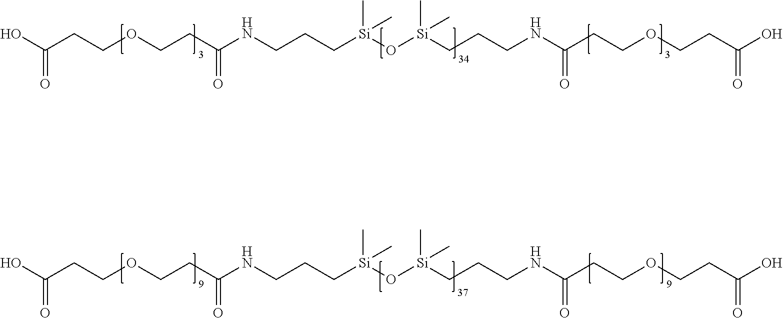

In some examples of the AB copolymer shown below including a PIB block as the B block and PEO as the A block, R is a moiety selected from the group consisting of amine group (—NH₂), thiol group (—SH), a dibenzocyclooctyne (DBCO) group, an azide group (—N₃), a biotin group, or a carboxyl group (—COOH); m=about 2 to about 100; n=about 2 to about 100; and L is a linker selected from the group consisting of an amide, a thioether (sulfide), a succinic group, a maleic group, an alkyl (such as methylene), an ether, or a products of a click reaction.

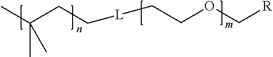

In another nonlimiting example shown below, n=13, m=3, R is a carboxyl group, and L is ethyl sulfide. In another nonlimiting example shown below, n=30, m=3, R is a carboxyl group, and L is ethyl sulfide.

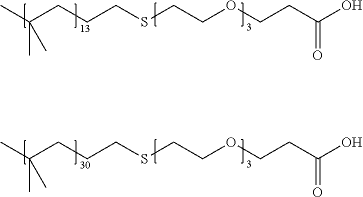

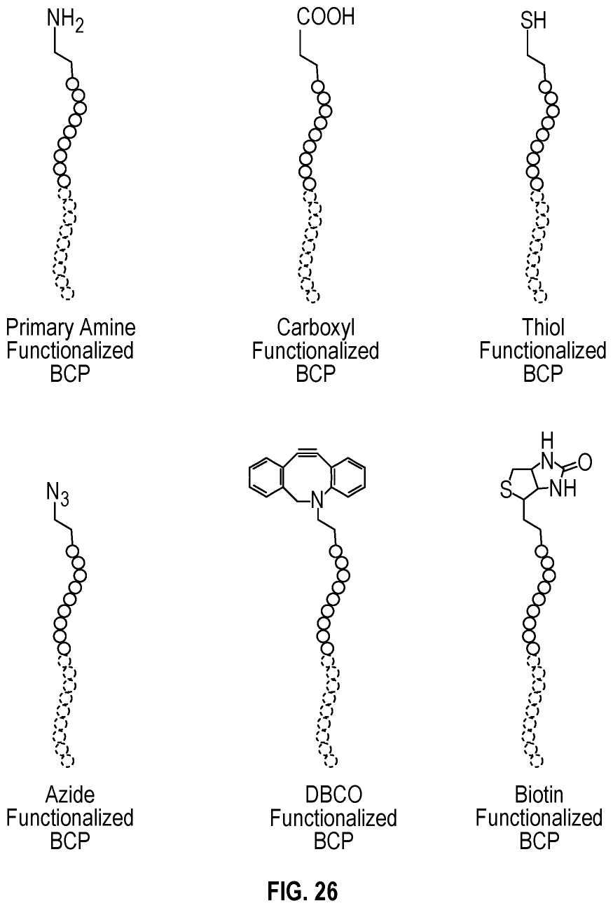

It will be appreciated that any suitable combination of reactive moieties may be used to couple molecules to amphiphilic molecules of a barrier. For example, any of the present reactive moieties (e.g., the reactive moiety 311, 511, or 611 of the amphiphilic molecules, such as AB, BAB, or ABA polymers), reactive moiety 321, reactive moiety 331, or reactive moiety 341 may be or include an amine group (—NH₂), thiol group (—SH), a dibenzocyclooctyne (DBCO) group, an azide group (—N₃), a biotin group, or a carboxyl group (—COOH). Nonlimiting examples of block copolymers including such reactive moieties are illustrated in FIG. 26; although FIG. 26 may illustrate AB block copolymers (BCPs), it should be appreciated that the illustrated moieties may be included in any suitable BCP, such as ABA or BAB. Table 1 includes various nonlimiting examples of pairs of reactive moieties that may be used to couple molecules to amphiphilic molecules of a barrier. For example, the reactive moieties may include an amine-NHS pair, an amine-imidoester pair, an amine-pentofluorophenyl ester pair, an amine-hydroxymethyl phosphine pair, an amine-carboxylic acid pair, a thiol-maleimide pair, a thiol-haloacetyl pair, a thiol-pyridyl disulfide pair, a thiol-thiosulfonate pair, a thiol-vinyl sulfone pair, an aldehyde-hydrazide pair, an aldehyde-alkoxyamine pair, a hydroxy-isocyanate pair, an azide-alkyne pair, an azide-phosphine pair, an azide-cyclooctyne pair, an azide-norbornene pair, a transcycloctene-tetrazine pair, a norbornene-tetrazine pair, an oxime, a SpyTag-SpyCatcher pair, a SNAP-tag-O⁶-benzylguanine pair, a CLIP-tag-O²-benzylcytosine pair, or a sortase coupling.

TABLE 1

| Bonding pair | Example moiety | Example moiety |
|---|---|---|
| amine-NHS | amine group, —NH₂ | N-Hydroxysuccinimide ester |
| | | 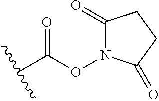 [SMILES: *C(=O)ON1C(=O)CCC1=O] |
| amine-imidoester | amine group, —NH₂ | imidoester |
| | | 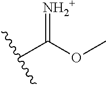 [SMILES: *C(=[NH2+])OC] |
| amine- | amine group, —NH₂ | pentofluorophenyl ester, |
| pentofluorophenyl ester | | 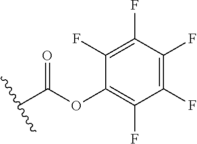 [SMILES: *C(=O)Oc1c(F)c(F)c(F)c(F)c1F] |
| amine- | amine group, —NH₂ | hydroxymethyl phosphine |
| hydroxymethyl phosphine | | 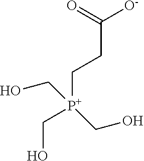 [SMILES: O=C([O-])CC[P+](CO)(CO)CO] |
| amine-carboxylic acid | amine group, —NH₂ | carboxylic acid group, —C(═O)OH (e.g., following activation of the carboxylic acid |
| thiol-maleimide | thiol, —SH | maleimide |
| | | 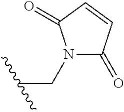 [SMILES: *CN1C(=O)C=CC1=O] |
| thiol-haloacetyl | thiol, —SH | haloacetyl (e.g., iodoacetyl or other |
| | | haloacetyl) |
| | | 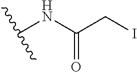 [SMILES: *NC(=O)CI] |
| thiol-pyridyl | thiol, —SH | pyridyl disulfide |
| disulfide | | 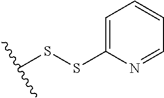 [SMILES: *SSc1ccccn1] |
| thiol-thiosulfonate | thiol, —SH | thiosulfonate |
| | | 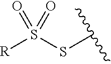 |
| thiol-vinyl sulfone | thiol, —SH | vinyl sulfone |
| | | 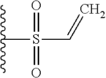 [SMILES: *S(=O)(=O)C=C] |
| aldehyde- | aldehyde, —C(═O)H | hydrazide |
| hydrazide | | 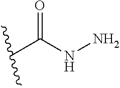 [SMILES: *C(=O)NN] |
| aldehyde- | aldehyde, —C(═O)H | alkoxyamine |
| alkoxyamine | | 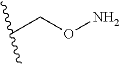 [SMILES: *CON] |
| hydroxy- | hydroxyl, —OH | isocyanate |
| isocyanate | | 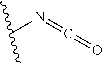 [SMILES: *N=C=O] |
| azide-alkyne | azide, —N₃ | alkyne |
| | | 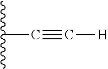 [SMILES: *C#C] |
| azide-phosphine | azide, —N₃ | phosphine, e.g.: |
| | | 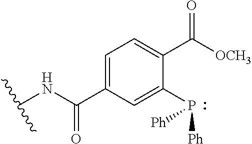 |
| azide- | azide, —N₃ | cyclooctyne, e.g. dibenzocyclooctyne |
| cyclooctyne | | (DBCO) |
| | | 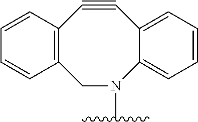 [SMILES: *N1Cc2ccccc2C#Cc2ccccc21] |
| | | or BCN (bicyclo[6.1.0]nonyne) |
| | | 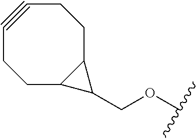 [SMILES: *OCC1C2CCC#CCCC21] |
| azide-norbornene | azine, —N₃ | norbornene |
| | | 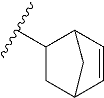 [SMILES: *C1CC2C=CC1C2] |
| transcyclooctene- | transcyclooctene | tetrazine, e.g., benzyl-methyltetrazine |
| tetrazine | 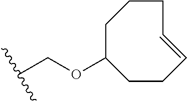 [SMILES: *COC1CC/C=C/CCC1] | 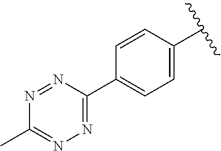 [SMILES: *c1ccc(-c2nnc(C)nn2)cc1] |
| norbornene- | norbornene | tetrazine, e.g. benzyl-tetrazine |
| tetrazine | 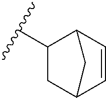 [SMILES: *C1CC2C=CC1C2] | 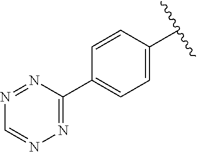 [SMILES: *c1ccc(-c2nncnn2)cc1] |
| oxime | aldehyde or ketone (e.g., amine | alkoxyamine |
| | cugroup or N-terminus of | |
| | polypeptide converted to an | |
| | aldehyde or ketone by pyroxidal | |
| | phosphate) | |
| SpyTag- | SpyTag: amino acid sequence | SpyCatcher amino acid sequence: |
| SpyCatcher | AHIVMVDAYKPTK | MKGSSHHHHHHVDIPTTENLYFQ |
| | | GAMVDTLSGLSSEQGQSGDMTIEE |
| | | DSATHIKFSKRDEDGKELAGATME |
| | | LRDSSGKTISTWISDGQVKDFYLY |
| | | PGKYTFVETAAPDGYEVATAITFT |
| | | VNEQGQVTVNGKATK |
| SNAP-tag-O⁶- | SNAP-tag (O-6-methylguanine- | O⁶-Benzylguanine |
| Benzylguanine | DNA methyltransferase) | 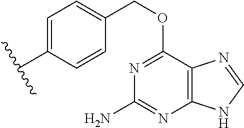 [SMILES: *c1ccc(COc2nc(N)nc3[nH]cnc23)cc1] |
| CLIP-tag-O²- | CLIP-tag (modified O-6- | O²-benzylcytosine |
| benzylcytosine | methylguanine-DNA methyltransferase) | 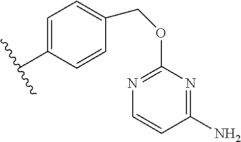 [SMILES: *c1ccc(COc2nccc(N)n2)cc1] |
| Sortase-coupling | -Leu-Pro-X-Thr-Gly | -Gly₍₃₋₅₎ |

In some examples, the reactive moieties may be reacted with one another using suitable permanent “conjugation” reactions, such as Cu(I)-catalyzed azide-alkyne cycloaddition (CuAAC), copper-free click chemistry, NHS coupling, or thiol-ene/yne coupling. Additionally, or alternatively, the reactive moieties may be reacted with one another using reversible coupling reactions, such as amine/aldehyde coupling (e.g., formation of hydrolysable imine linkage) or cross-linking agents optionally including a cleavable group such as urea, disulfide bonds, or nitrobenzyl-bearing compounds. Nonlimiting examples of molecules including reactive moieties are illustrated below:

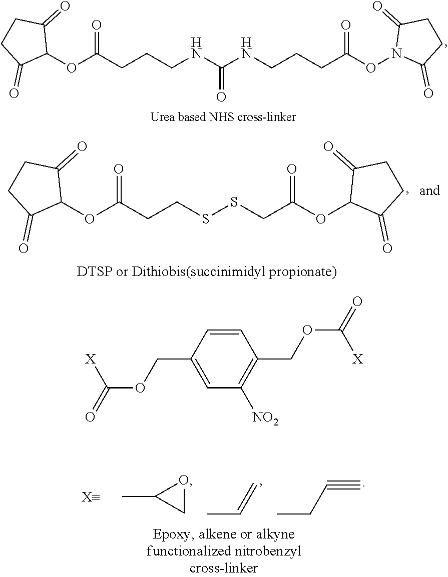

In some examples, a reaction between reactive moieties in a manner such as described with reference to FIG. 3B-3C, 3D-3E, or 3F-3G includes an azide-alkyne [3+2] cyclo-addition, which may be referred to as a Huisgen cyclo-addition. A first one of the reactive moieties may include azide (N₃), and the reactive moiety that reacts with that reactive moiety may include a dibenzocyclooctyne (DBCO) having the structure:

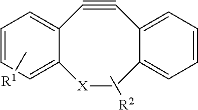

wherein one of R₁ and R₂ is H and the other is a linkage to the barrier (e.g., to the amphiphilic molecule or the product of an earlier reaction) or a linker to another reactive moiety (e.g., where the reactive moiety is part of a multifunctional molecule in solution); and wherein X is CH₂, O, S, or NH if R₂ is not directly coupled to X, or wherein X is CH or N if R₂ is directly coupled to X. The azide may react with the dibenzocyclooctyne to form a cycloadduct having the structure:

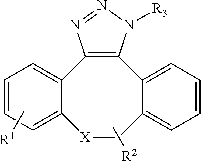

where R₃ is a linkage to the molecule in solution (if the amphiphilic molecule or the product of the earlier reaction includes the DBCO) or to the amphiphilic molecule or the product of the earlier reaction includes the DBCO (if the molecule in solution includes the DBCO). Such reaction, or any other azide-alkyne [3+2] cyclo-addition reaction, optionally may be performed without the use of catalyst. Additionally, or alternatively, the azide-alkyne [3+2] cyclo-addition reaction optionally may be promoted using heat (e.g., from light).

It will be appreciated that the DBCO represents a non-limiting example of an alkyne that may be used in an azide-alkyne [3+2] cyclo-addition reaction between reactive moieties. It will also be appreciated that azide-alkyne [3+2] cyclo-addition reactions represent a non-limiting example of a suitable reaction between reactive moieties to covalently couple molecules to the amphiphilic molecules of the barrier. Other example alkynes that may be used in an azide-alkyne [3+2] cyclo-addition reaction between first moieties 113 and second moieties 123 include other strained cyclooctynes such as bicyclononyne (BCN) or a derivative thereof, difluorocyclooctyne (DIFO) or a derivative thereof, dibenzocyclooctyne (DIBO) or a derivative thereof, and the like. Some nonlimiting examples of strained cyclooctynes that may be used in an azide-alkyne [3+2] cyclo-addition reaction between reactive moieties include the following, in which R represents a connection to the barrier or to the molecule in solution:

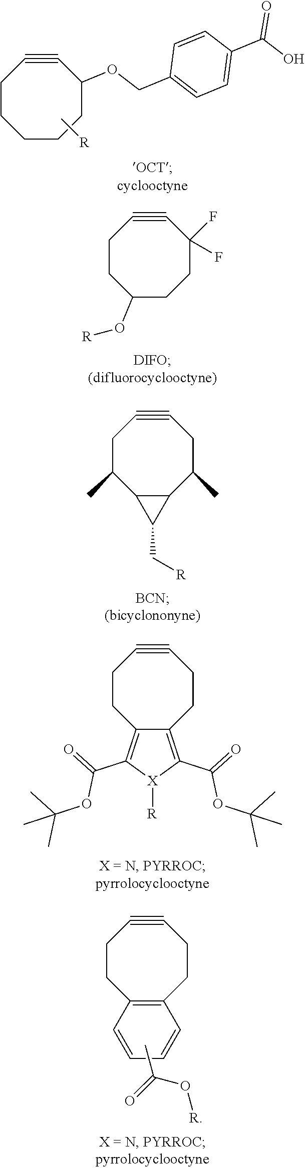

For further details regarding example reactions between cycloalkynes and azides that may be adapted for use in the present devices, compositions, and methods and methods, see Dommerholt et al., “Strain-promoted 1,3-dipolar cycloaddition of cycloalkynes and organic azides,” Top. Curr. Chem. (Z) 374: 16, 20 pages (2016), the entire contents of which are incorporated by reference herein. However, any reactive moieties that form bonds through cycloaddition reactions may be used, such as aryl azides and pentafluoro alkynes. It will also be appreciated that reactive moieties that form bonds through other types of addition reactions, such as the thermally driven reaction of a primary amine with an epoxy group, suitably may be used.

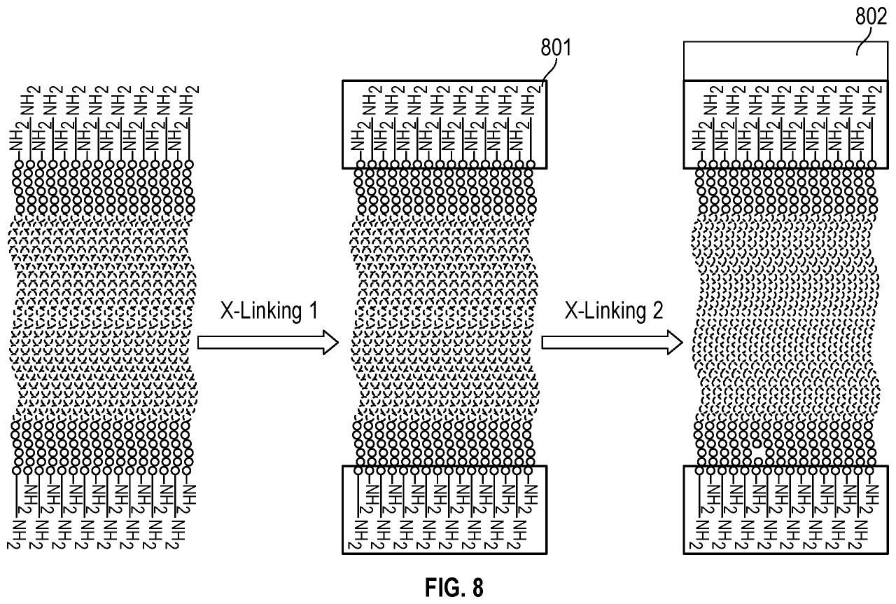

For example, FIG. 8 schematically illustrates use of a first set of example reactive moieties to couple molecules to a barrier. In the nonlimiting example illustrated in FIG. 8, the barrier includes amphiphilic molecules (e.g., AB, BAB, or ABA copolymer), which include amine groups (—NH₂) as reactive moieties. The barrier is contacted with a solution including reactive molecules, corresponding to multifunctional molecules 320 in FIG. 3B, that include N-hydroxysuccinimide (NHS) as the reactive moiety. Illustratively, the reactive molecules may be selected from the group consisting of:

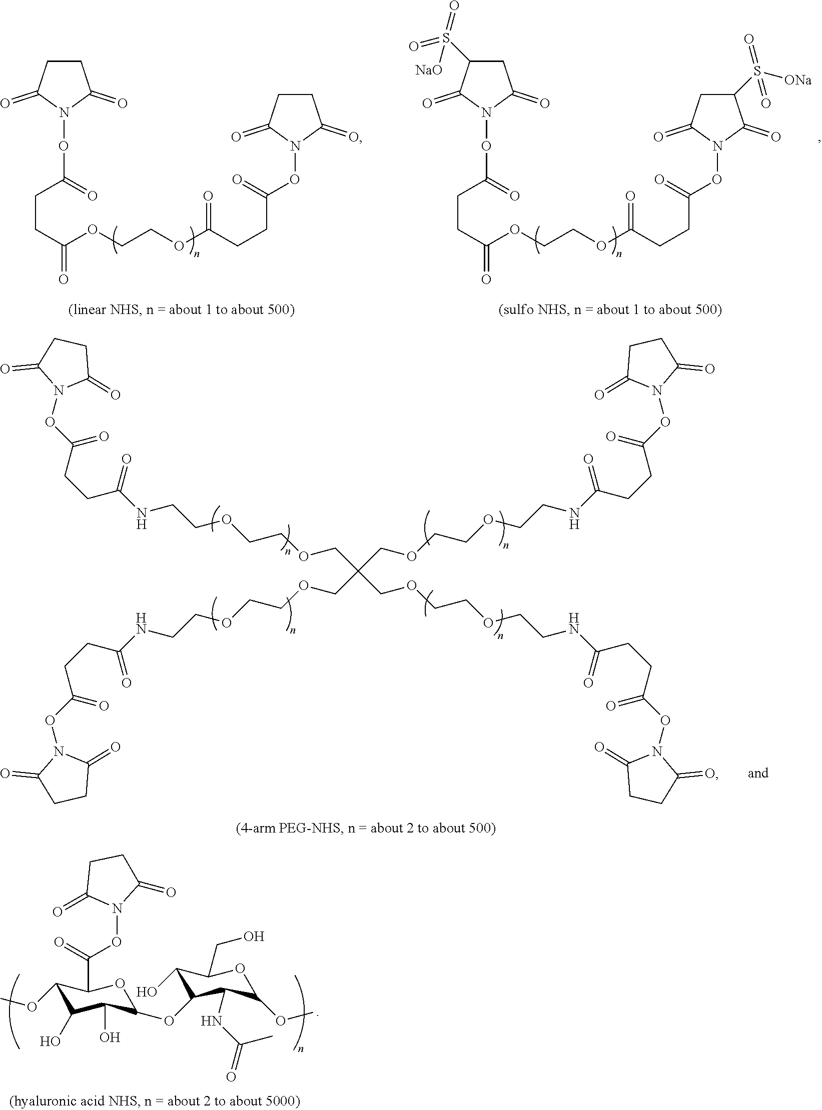

The NHS and NH₂ moieties react with one another in a reaction referred to in FIG. 8 as “X-linking 1” to form layer 801. In a manner similar to that described with reference to FIG. 3C, layer 801 may include a first reaction product, corresponding to first reaction product 320′ in FIG. 3C, in which one of the NHS moieties reacts with an NH₂ moiety and the other NHS moiety remains available for a subsequent reaction. Optionally, layer 801 also or alternatively may include a second reaction product, corresponding to second reaction product 320″ in FIG. 3C, in which at least two of the NHS moieties react with corresponding NH₂ moieties and thus cross-link the amphiphilic molecules.

The available NHS moieties of the reaction products within layer 801 optionally then are reacted with multifunctional (e.g., bifunctional) reactive molecules, corresponding to multifunctional molecules 330 in FIG. 3D, that include NH₂ as the reactive moiety. Illustratively, the reactive molecules may be selected from the group consisting of:

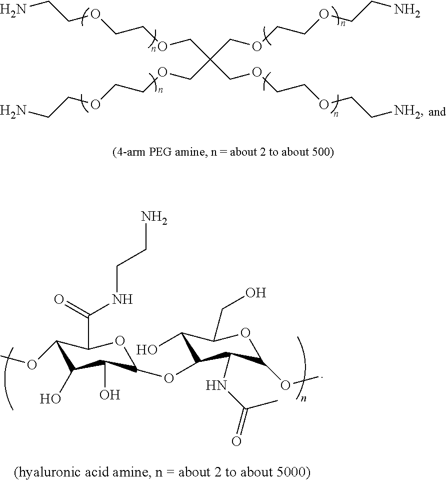

The NHS and NH₂ moieties react with one another in a reaction referred to in FIG. 8 as “X-linking 2” to form layer 802. In a manner similar to that described with reference to FIG. 3E, layer 802 may include a first reaction product, corresponding to first reaction product 330′ in FIG. 3E, in which one of the NH₂ moieties reacts with an NHS moiety and the other NH₂ moiety remains available for a subsequent reaction. Optionally, layer 802 also or alternatively may include a second reaction product, corresponding to second reaction product 330″ in FIG. 3E, in which at least two of the NH₂ moieties react with corresponding NHS moieties and thus cross-link the amphiphilic molecules.

The available NH₂ moieties of the reaction products within layer 802 optionally then are reacted with multifunctional (bifunctional) reactive molecules, corresponding to multifunctional molecules 340 in FIG. 3F, that include NHS as the reactive moiety. These reactive molecules may be the same as, or different than, the reactive molecules which correspond to multifunctional molecules 320 in FIG. 3B. Illustratively, the reactive molecules may be selected from a similar group of molecules as used during “X-linking 1”, e.g., the group consisting of:

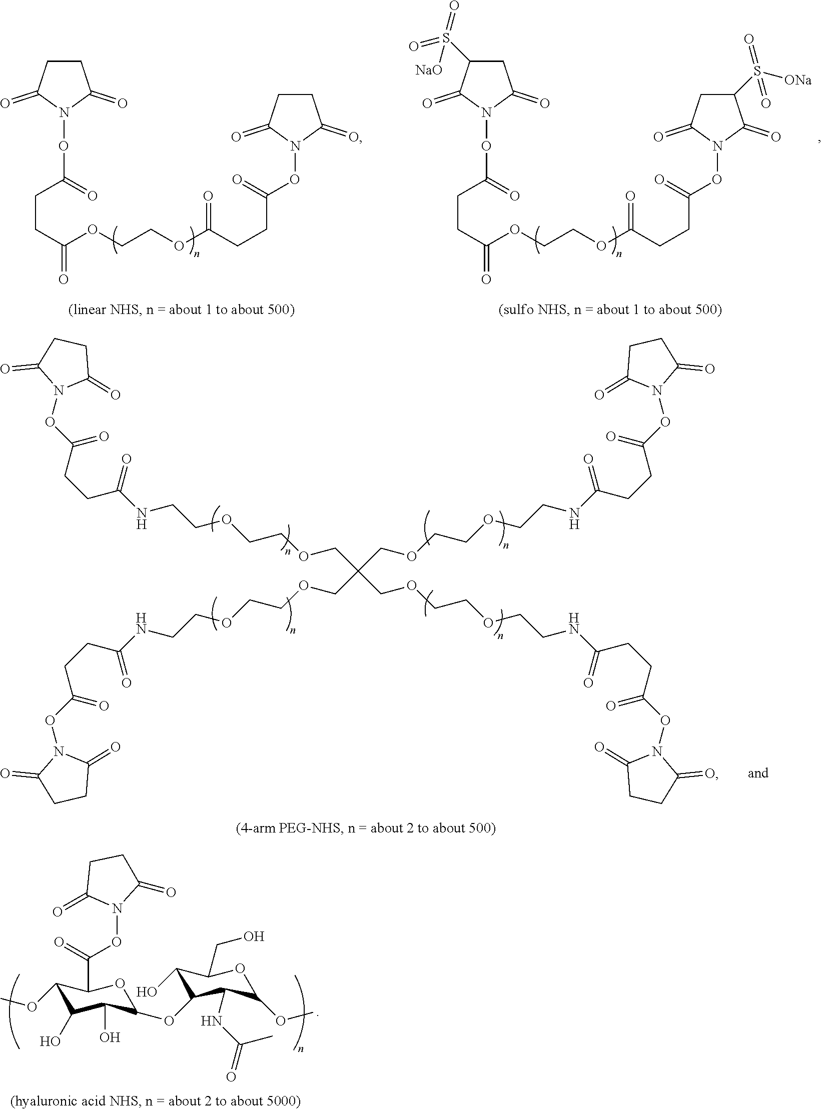

The NHS and NH₂ moieties may react with one another in a reaction similar to that referred to in FIG. 8 as “X-linking 1” to form another layer coupled to layer 802. In a manner similar to that described with reference to FIG. 3G, this additional layer may include a first reaction product, corresponding to first reaction product 340′ in FIG. 3G, in which one of the NHS moieties reacts with an NH₂ moiety and the other NHS moiety remains available for a subsequent reaction. Optionally, the additional layer also or alternatively may include a second reaction product, corresponding to second reaction product 340″ in FIG. 3G, in which at least two of the NHS moieties react with corresponding NH₂ moieties and thus cross-link the amphiphilic molecules. Any available NHS moieties of the reaction products within the additional layer optionally then are reacted with multifunctional (e.g., bifunctional) reactive molecules that include NH₂ moieties. Such process of adding layers may be repeated as many times as desired using any suitable molecules in solution.

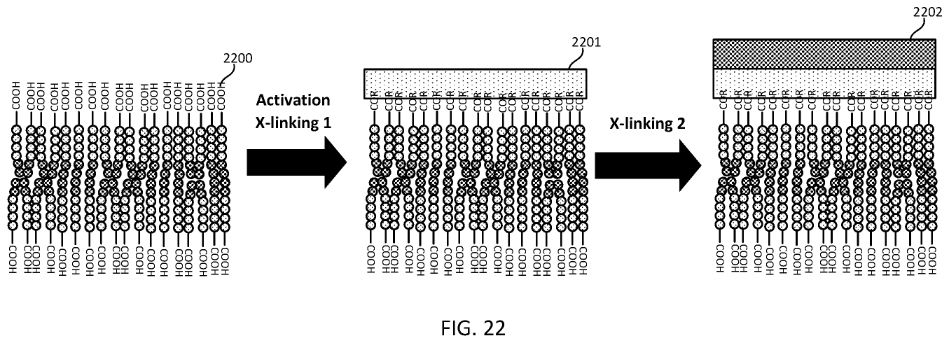

In another example, FIG. 22 schematically illustrates use of a second set of example reactive moieties to couple molecules to a barrier. In the nonlimiting example illustrated in FIG. 22, barrier 2200 includes amphiphilic molecules (e.g., AB, BAB, or ABA copolymer, which may be configured in a manner such as described elsewhere herein), which include carboxyl groups (—COOH) as the first reactive moieties. The barrier 2200 is contacted with a solution including activating molecules and reactive molecules. The reactive molecules may be multifunctional (e.g., may include multiple second reactive moieties), and may include a linear, branched, or dendritic molecule such as an oligomer or polymer. In some examples, the second reactive moieties include amine groups (—NH₂) or hydroxyl groups (—OH). Illustratively, the reactive molecules may be

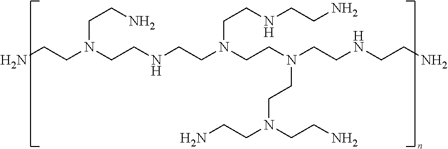

a functionalized branched PEG (e.g., 4-arm PEG-NH₂), hyaluronic acid (e.g., HA-NH₂), or any other hydrophilic oligomeric or polymeric structure including a plurality of nucleophilic groups able to react with carboxylic acids or an activated ester (such as, but not limited to, primary amines).

Illustratively, the activating molecules may include (4-(4,6-dimethoxy-1,3,5-triazin-2-yl)-4-methyl-morpholimium chloride (DMTMM)), 1-ethyl-3-(3′-(dimethylamino)propyl)carbodiimide (EDC), EDC in combination with N-hydroxyl succinimide (NHS), ethyl 2-cyano-2(hydroxylamino)acetate uranium salt (COMU), N,N′-carbonyldiimidazole (CDI), or 0-(1,2-dihydro-2-oxo-1-pyridyl-N,N,N′,N′-tetramethyluronium tetrafluoroborate (TPTU). Nonlimiting examples of such molecules are illustrated below:

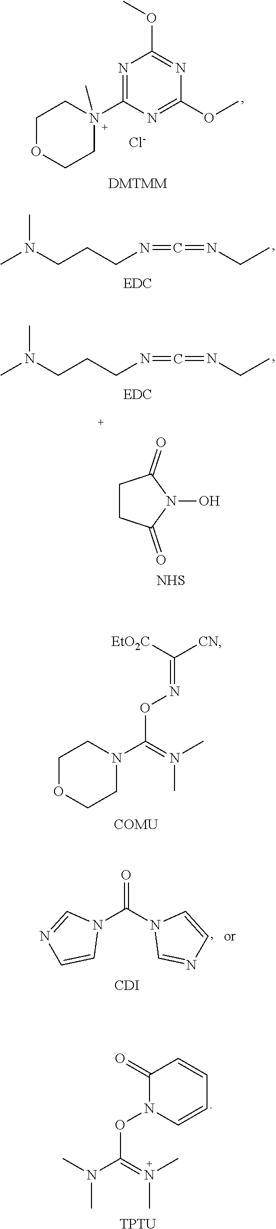

The activating molecules and carboxyl moieties react with one another in a reaction referred to in FIG. 22 as “Activation”, resulting in an activated carboxyl moiety. In examples in which DMTMM is used for activation, the reaction also releases a molecule of N-methylmorpholinium (NMM). The activated carboxyl moieties and second reactive moieties then react with one another in a reaction referred to in FIG. 22 as “X-linking 1” to form layer 2201. The second reactive moiety (such as an amine or a hydroxyl/alkoxide or a thiol/thiolate group) acts as a nucleophile and performs a nucleophilic attack on the activated carboxyl moiety of the block copolymer in order to form a bond. In some examples, the reaction also releases a molecule, illustratively of 4,6-dimethoxy-1,3,5-triazin-2-ol in examples in which DMTMM is used for activation. In a manner similar to that described with reference to FIG. 3C, layer 2201 illustrated in FIG. 22 may include a first reaction product, corresponding to product 320′ of FIG. 3C, in which one of the second reactive moieties reacts with an activated carboxyl moiety and other second reactive moiet(ies) remain available for a subsequent reaction. Optionally, layer 2201 also or alternatively may include a second reaction product, corresponding to product 320″ of FIG. 3C, in which more than one of the second reaction moieties react with corresponding activated carboxyl moieties and thus cross-link the amphiphilic molecules.

The available second reactive moieties of the reaction products within layer 2201 optionally then are reacted with multifunctional reactive molecules, corresponding to molecules 330 of FIG. 3D, that include a third reactive moiety. In some examples, the third reactive moiety may include NHS. Illustratively, the reactive molecules may be selected from the group consisting of:

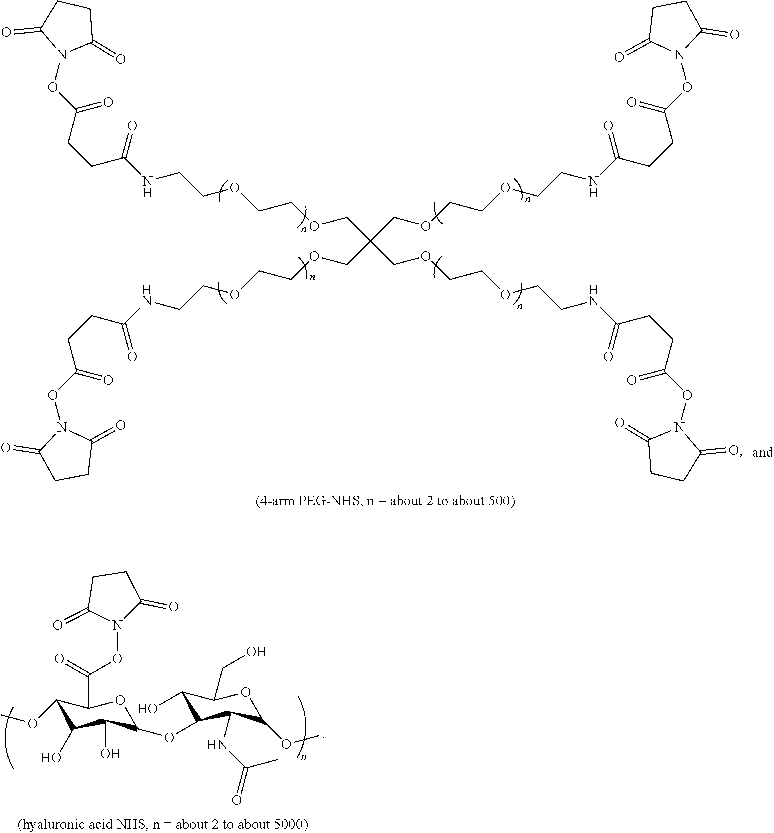

The third reactive moieties and second reactive moieties may react with one another in a reaction referred to in FIG. 8 as “X-linking 2” to form layer 2202 which is coupled to layer 2201. For example, the NHS moieties may react with amine moieties that were installed in the X-linking 1 step. In a manner similar to that described with reference to FIG. 3E, layer 2202 may include a first reaction product, corresponding to 330′ of FIG. 3E, in which more than one of the third reactive moieties react with corresponding second reactive moieties and thus cross-link the amphiphilic molecules.

The available third reactive moieties of the reaction products within layer 2202 optionally then are reacted with multifunctional reactive molecules, corresponding to molecules 340 of FIG. 3F, that include a fourth reactive moiety. The fourth reactive moiety may be the same as, or different than, the second reactive moiety. In some examples, the fourth reactive moiety may be NH₂. Illustratively, the reactive molecules may be selected from a similar group of molecules as used during “X-linking 1”, e.g., the group consisting of:

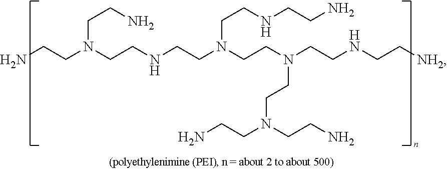

a functionalized branched PEG (e.g., 4-arm PEG-NH₂), hyaluronic acid (e.g., HA-NH₂), or any other hydrophilic oligomeric or polymeric structure including a plurality of nucleophilic groups able to react with carboxylic acids or an activated ester (such as, but not limited to, primary amines).

The fourth reactive moieties and third reactive moieties may react with one another in a reaction similar to that referred to in FIG. 22 as “X-linking 1” to form another layer coupled to layer 2202. In a manner similar to that described with reference to FIG. 3G, this additional layer may include a first reaction product, corresponding to product 340′ of FIG. 3G, in which one of the fourth reactive moieties reacts with a third reactive moiety and one fourth reactive moiety remains available for a subsequent reaction. Optionally, the additional layer also or alternatively may include a second reaction product, corresponding to product 340″ of FIG. 3G, in which both of the fourth reactive moieties react with corresponding third reactive moieties and thus cross-link the amphiphilic molecules. Any available fourth reactive moieties of the reaction products within the additional layer optionally then are reacted with reactive molecules that include fifth reactive moieties. In some examples, the fifth reactive moieties may include NHS. Such process of adding layers may be repeated as many times as desired using any suitable molecules in solution.

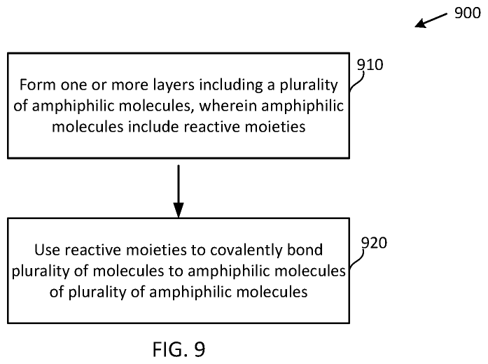

Accordingly, it will be appreciated that a wide variety of amphiphilic molecules and a wide variety of reactive moieties may be used to generate barriers that are stabilized using covalent bonds to molecules, e.g., for use in a nanopore device such as described with reference to FIG. 1. FIG. 9 illustrates an example flow of operations in a method 900 for forming a barrier including molecules covalently bonded to amphiphilic molecules. Method 900 may include forming one or more layers including a plurality of amphiphilic molecules, wherein the amphiphilic molecules include reactive moieties (operation 910). In some examples, operation 910 may include forming first and second layers respectively including first and second pluralities of amphiphilic molecules, wherein the amphiphilic molecules include reactive moieties. For example, barrier 101 may include molecules of block copolymers (e.g., AB, ABA, or BAB), the hydrophilic “A” blocks of which may be coupled to reactive moieties (e.g., 311, 511, or 611, respectively) in a manner such as described with reference to FIG. 3A-3H, 4, 5, 6, 8, or 22.

Illustratively, the block copolymer is an AB diblock copolymer which forms first and second layers, such as described with reference to FIGS. 2A-2B and 7B. As such, the barrier may have a thickness of approximately 2A+2B. In one nonlimiting example of such a diblock copolymer, the hydrophobic block may be polybutadiene (PBd). Alternatively, the block copolymer may be an ABA triblock copolymer having two hydrophilic blocks and one hydrophobic block, such as described with reference to FIGS. 5 and 7A. As such, in various examples the barrier may form a first layer, or may form first and second layers, or may form partially a single layer and partially a bilayer. In any such examples, the barrier may have a thickness of approximately 2A+B. In one nonlimiting example of such a triblock copolymer, the hydrophobic block is poly(isobutylene) (PIB) or PDMS. In yet another alternative, the block copolymer is a BAB triblock copolymer having two hydrophobic blocks and one hydrophilic block. As such, the barrier may form first and second layers and may have a thickness of approximately A+2B.

Optionally, the barrier formed in operation 910 may be suspended by a barrier support defining an aperture. The barrier may include one or more layers suspended across the aperture, and may be formed using any suitable combination of operations provided herein or otherwise known in the art. For example, forming the barrier may include “painting” as known in the art. Known techniques for painting barriers that are suspended by barrier supports include brush painting (manual), mechanical painting (e.g., using stirring bar), and bubble painting (e.g., using flow through the device).

Method 900 illustrated in FIG. 9 also may include using the reactive moieties to covalently bond a plurality of molecules to the amphiphilic molecules of the plurality of amphiphilic molecules (operation 920). In some examples, operation 920 may include activating the first reactive moieties using an activating molecule, and covalently bonding the first plurality of molecules to the activated first reactive moieties. For example, the molecules may be provided in a solution in contact with the plurality of amphiphilic molecules, and may include reactive moieties that react with the reactive moieties of the amphiphilic molecules, e.g., in a manner such as described with reference to FIGS. 3B-3C and 22. In some examples, the reaction products may retain one or more reactive moieties via which additional molecules may be coupled to the barrier, e.g., in a manner such as described with reference to FIG. 3D-3G and 22. Additionally, or alternatively, the reaction products may cross-link the amphiphilic molecules, e.g., in a manner such as described with reference to FIGS. 3D-3G and 22. Optionally, a nanopore may be inserted into the barrier at any suitable time, e.g., before any of the reactions described herein, or after any of the reactions described herein.

It will further be appreciated that the present barriers may be used in any suitable device or application. For example, FIG. 18 schematically illustrates a cross-sectional view of an example use of the composition and device of FIG. 1. Device 100 illustrated in FIG. 18 may be configured to include fluidic well 100′, barrier 101 which may have a configuration such as described elsewhere herein, first and second fluids 120, 120′, and nanopore 110 in a manner such as described with reference to FIG. 1. In the nonlimiting example illustrated in FIG. 18, second fluid 120′ optionally may include a plurality of each of nucleotides 121, 122, 123, 124, e.g., G, T, A, and C, respectively. Each of the nucleotides 121, 122, 123, 124 in second fluid 120′ optionally may be coupled to a respective label 131, 132, 133, 134 coupled to the nucleotide via an elongated body (elongated body not specifically labeled). Optionally, device 100 further may include polymerase 105. As illustrated in FIG. 18, polymerase 105 may be within the second composition of second fluid 120′. Alternatively, polymerase 105 may be coupled to 110 nanopore or to barrier 101, e.g., via a suitable elongated body (not specifically illustrated). Device 100 optionally further may include first and second polynucleotides 140, 150 in a manner such as illustrated in FIG. 18. Polymerase 105 may be for sequentially adding nucleotides of the plurality to the first polynucleotide 140 using a sequence of the second polynucleotide 150. For example, at the particular time illustrated in FIG. 18, polymerase 105 incorporates nucleotide 122 (T) into first polynucleotide 140, which is hybridized to second polynucleotide 150 to form a duplex. At other times (not specifically illustrated), polymerase 105 sequentially may incorporate other of nucleotides 121, 122, 123, 124 into first polynucleotide 140 using the sequence of second polynucleotide 150.

Circuitry 180 illustrated in FIG. 18 may be configured to detect changes in an electrical characteristic of the aperture responsive to the polymerase sequentially adding nucleotides of the plurality to the first polynucleotide 140 using a sequence of the second polynucleotide 150. In the nonlimiting example illustrated in FIG. 18, nanopore 110 may be coupled to permanent tether 1810 which may include head region 1811, tail region 1812, elongated body 1813, reporter region 1814 (e.g., an abasic nucleotide), and moiety 1815. Head region 1811 of tether 1810 is coupled to nanopore 110 via any suitable chemical bond, protein-protein interaction, or any other suitable attachment that is normally irreversible. Head region 1811 can be attached to any suitable portion of nanopore 110 that places reporter region 1814 within aperture 1813 and places moiety 1815 sufficiently close to polymerase 105 so as to interact with respective labels 131, 132, 133, 134 of nucleotides 121, 122, 123, 124 that are acted upon by polymerase 105. Moiety 1815 respectively may interact with labels 131, 132, 133, 134 in such a manner as to move reporter region 1814 within aperture 113 and thus alter the rate at which salt 160 moves through aperture 113, and thus may detectably alter the electrical conductivity of aperture 113 in such a manner as to be detected by circuitry 180. For further details regarding use of permanent tethers coupled to nanopores to sequence polynucleotides, see U.S. Pat. No. 9,708,655, the entire contents of which are incorporated by reference herein.

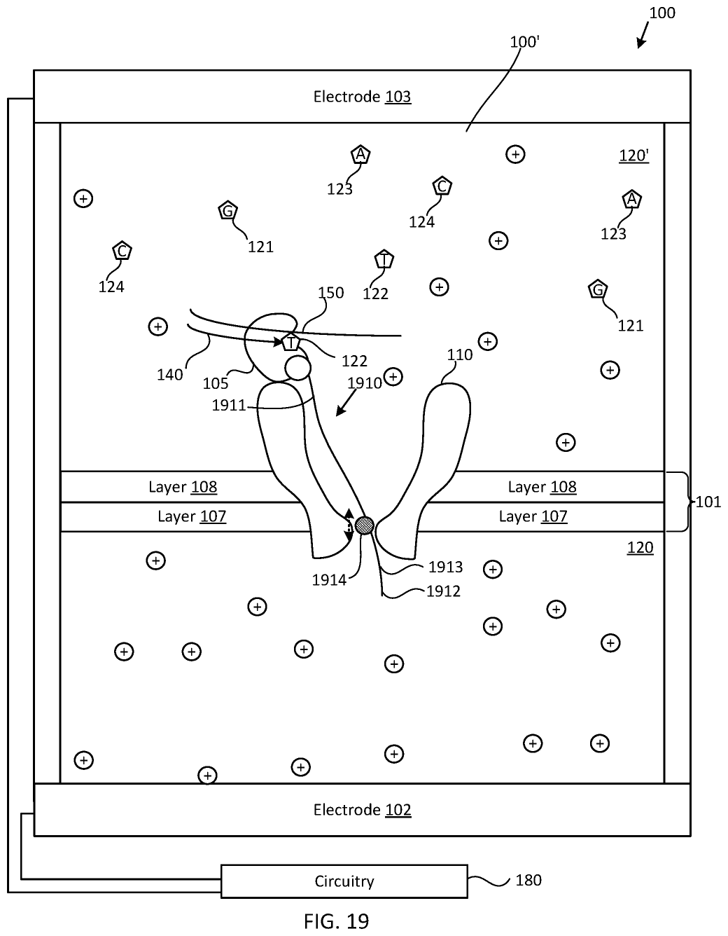

FIG. 19 schematically illustrates a cross-sectional view of another example use of the composition and device of FIG. 1. As illustrated in FIG. 19, device 100 may include fluidic well 100′, barrier 101 which may have a configuration such as described elsewhere herein, first and second fluids 120, 120′, nanopore 110, and first and second polynucleotides 140, 150, all of which may be configured similarly as described with reference to FIG. 18. In the nonlimiting example illustrated in FIG. 19, nucleotides 121, 122, 123, 124 need not necessarily be coupled to respective labels. Polymerase 105 may be coupled to nanopore 110 and may be coupled to permanent tether 1910 which may include head region 1911, tail region 1912, elongated body 1913, and reporter region 1914 (e.g., an abasic nucleotide). Head region 1911 of tether 1910 is coupled to polymerase 105 via any suitable chemical bond, protein-protein interaction, or any other suitable attachment that is normally irreversible. Head region 1911 can be attached to any suitable portion of polymerase 105 that places reporter region 1914 within aperture 113. As polymerase 105 interacts with nucleotides 121, 122, 123, 124, such interactions may cause polymerase 105 to undergo conformational changes. Such conformational changes may move reporter region 1914 within aperture 113 and thus alter the rate at which salt 160 moves through aperture 113, and thus may detectably alter the electrical conductivity of aperture 113 in such a manner as to be detected by circuitry 180. For further details regarding use of permanent tethers coupled to polymerases to sequence polynucleotides, see U.S. Pat. No. 9,708,655, the entire contents of which are incorporated by reference herein.

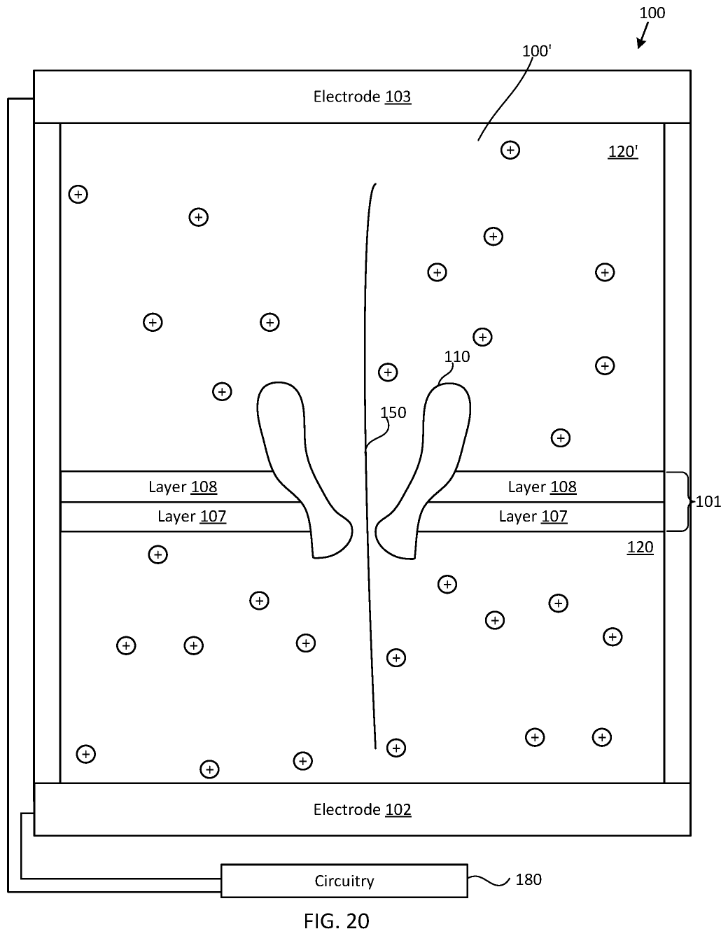

FIG. 20 schematically illustrates a cross-sectional view of another example use of the composition and device of FIG. 1. As illustrated in FIG. 20, device 100 may include fluidic well 100′, barrier 101 which may have a configuration such as described elsewhere herein, first and second fluids 120, 120′, and nanopore 110 all of which may be configured similarly as described with reference to FIG. 18. In the nonlimiting example illustrated in FIG. 20, polynucleotide 150 is translocated through nanopore 110 under an applied force, e.g., a bias voltage that circuitry 180 applies between electrode 102 and electrode 103. As bases in polynucleotide 150 pass through nanopore 110, such bases may alter the rate at which salt 160 moves through aperture 113, and thus may detectably alter the electrical conductivity of aperture 113 in such a manner as to be detected by circuitry 180. For further details regarding use of nanopores to sequence polynucleotides being translocated therethrough, see U.S. Pat. No. 5,795,782, the entire contents of which are incorporated by reference herein.

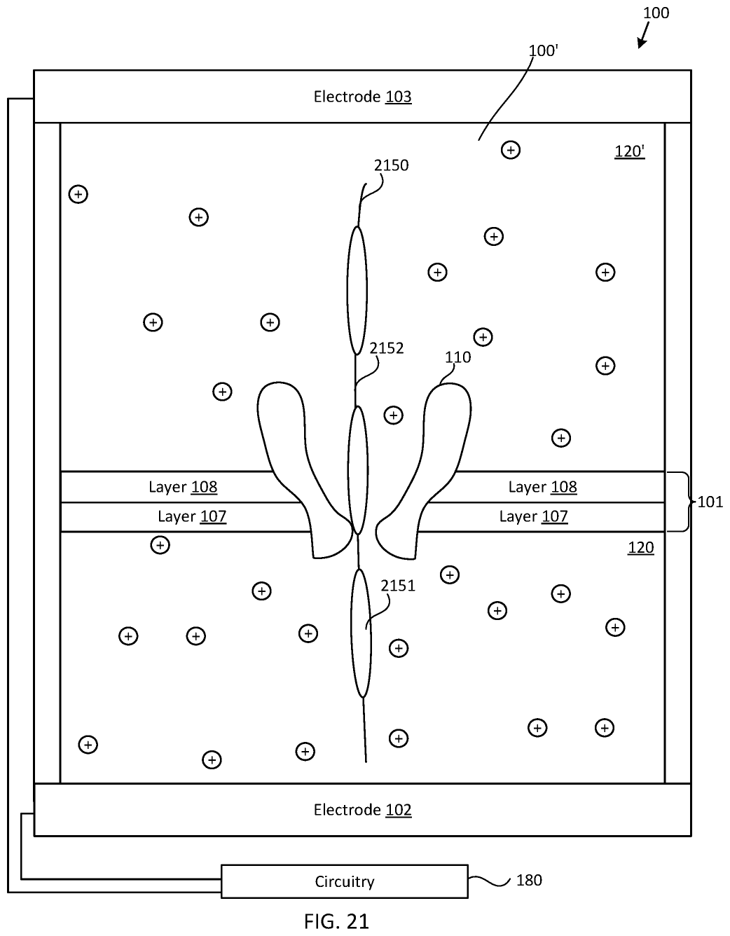

FIG. 21 schematically illustrates a cross-sectional view of another example use of the composition and device of FIG. 1. As illustrated in FIG. 21, device 100 may include fluidic well 100′, barrier 101 which may have a configuration such as described elsewhere herein, first and second fluids 120, 120′, and nanopore 110 all of which may be configured similarly as described with reference to FIG. 18. In the nonlimiting example illustrated in FIG. 21, surrogate polymer 2150 is translocated through nanopore 110 under an applied force, e.g., a bias voltage that circuitry 180 applies between electrode 102 and electrode 103. As used herein, a “surrogate polymer” is intended to mean an elongated chain of labels having a sequence corresponding to a sequence of nucleotides in a polynucleotide. In the example illustrated in FIG. 21, surrogate polymer 2150 includes labels 2151 coupled to one another via linkers 2152. An XPANDOMER™ is a particular type of surrogate polymer developed by Roche Sequencing, Inc. (Pleasanton, CA). XPANDOMERS™ may be prepared using Sequencing By eXpansion™ (SBX™, Roche Sequencing, Pleasanton CA). In Sequencing by eXpansion™, an engineered polymerase polymerizes xNTPs which include nucleobases coupled to labels via linkers, using the sequence of a target polynucleotide. The polymerized nucleotides are then processed to generate an elongated chain of the labels, separated from one another by linkers which are coupled between the labels, and having a sequence that is complementary to that of the target polynucleotide. For example descriptions of XPANIDOIVIIERS™, linkers (tethers), labels, engineered polymerases, and methods for SBX™, see the following patents, the entire contents of each of which are incorporated by reference herein: U.S. Pat. Nos. 7,939,249, 8,324,360, 8,349,565, 8,586,301, 8,592,182, 9,670,526, 9,771,614, 9,920,386, 10,457,979, 10,676,782, 10,745,685, 10,774,105, and 10,851,405.

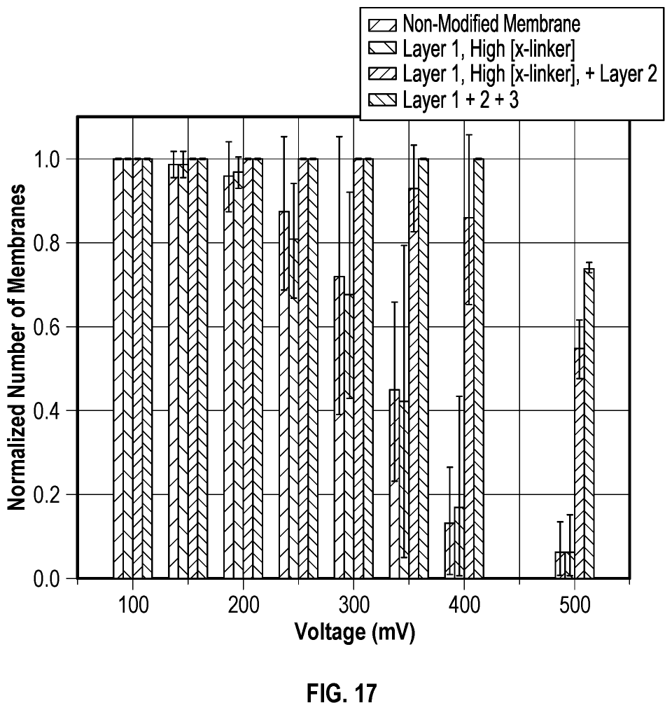

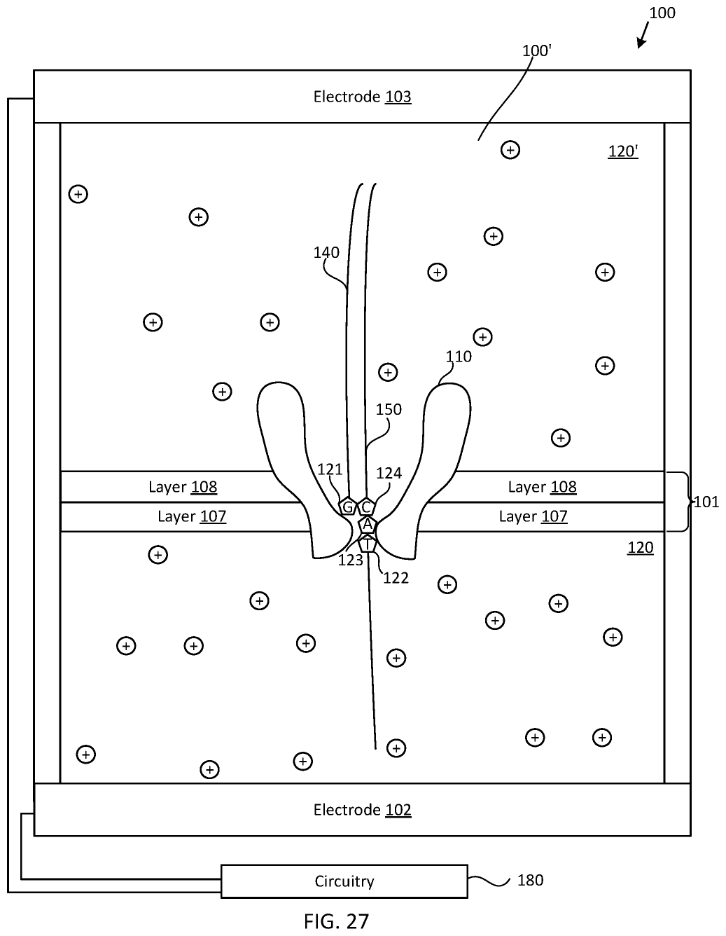

FIG. 27 schematically illustrates a cross-sectional view of another example use of the composition and device of FIG. 1. As illustrated in FIG. 17, device 100 may include fluidic well 100′, barrier 101 which may have a configuration such as described herein (that is, barrier 101 optionally may be suspended using a barrier support, and may include any AB, ABA, or BAB copolymer which is cross-linked in a manner such as provided herein), first and second fluids 120, 120′, and nanopore 110 all of which may be configured similarly as described elsewhere herein. In the nonlimiting example illustrated in FIG. 27, a duplex between polynucleotide 140 and polynucleotide 150 is located within nanopore 110 under an applied force, e.g., a bias voltage that circuitry 180 applies between electrode 102 and electrode 103. A combination of bases in the double-stranded portion (here, the base pair GC 121, 124 at the terminal end of the duplex) and bases in the single-stranded portion of polynucleotide 150 (here, bases A and T 123, 122) may alter the rate at which salt 160 moves through aperture 113, and thus may detectably alter the electrical conductivity of aperture 113 in such a manner as to be detected by circuitry 180. For further details regarding use of nanopores to sequence polynucleotides being translocated therethrough, see US Patent Publication No. 2023/0090867 to Mandell et al., the entire contents of which are incorporated by reference herein.

### WORKING EXAMPLES

The following examples are intended to be purely illustrative, and not limiting of the present invention.

### Example 1

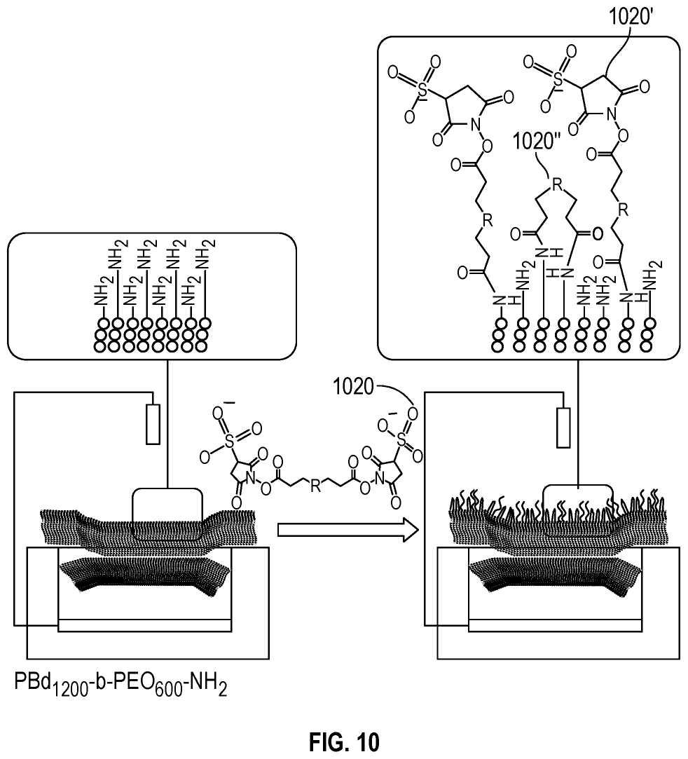

To assess barrier stability improvement provided by reactions with molecules, barrier resistance to breakdown voltage was evaluated using an experimental setup such as illustrated in FIG. 10. After barrier painting of the functionalized diblock copolymer material PBd₁₂₀₀-b-PEO₆₀₀-NH₂, a first layer was formed by reacting the NH₂ moieties with the following bifunctional sulfo-NHS material 1020, in which n=1 in this example:

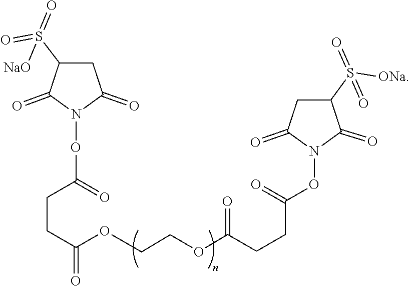

As illustrated in FIG. 10, the products of the reaction potentially included a mixture of a first product 1020′ in which an NHS moiety remained available for further reaction, and a second product 1020″ in which both NHS moieties reacted with the NH₂ moieties to crosslink the diblock copolymer.

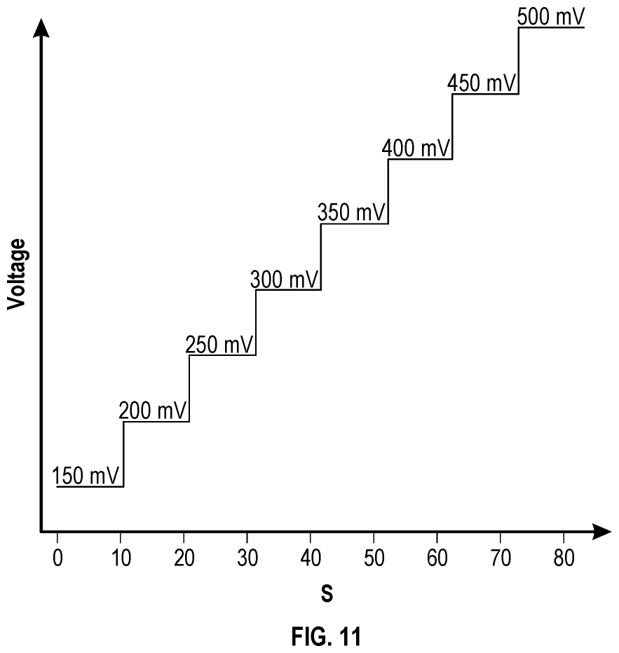

FIG. 11 illustrates the voltage breakdown waveform used to assess polymeric barrier stability. Barrier stability was quantified as the percentage of barriers remaining at the end of each step of the voltage ramp illustrated. The voltage ramp was stepped in 50 mV steps from 150 mV to 500 mV, as shown in FIG. 11. Each step lasted for 10 seconds. Nanopore insertion was represented as the number of successful single nanopore insertions during each individual experiment with a maximum of 16 nanopores per experiment. The barriers were generated under standard buffer conditions (1M KCl, 50 mM HEPES, pH=7.4).

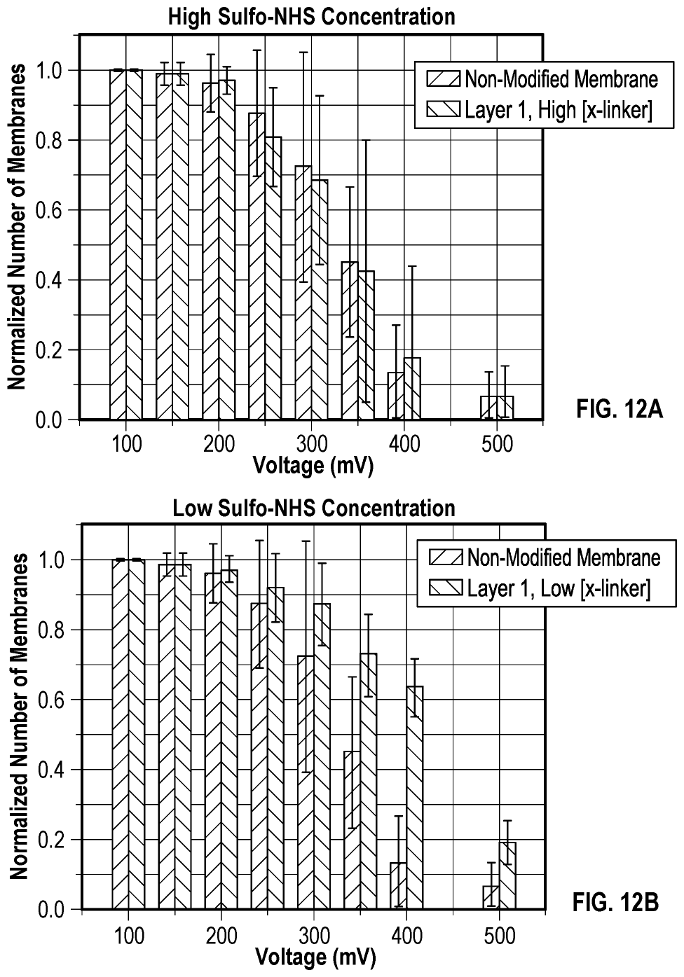

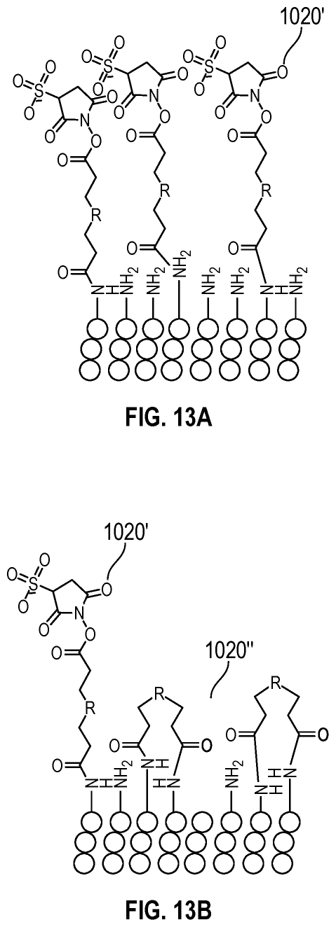

FIGS. 12A and 12B are plots of the measured barrier stability for barriers reacted with molecules under different conditions. More specifically, FIG. 12A illustrates the measured barrier stability for barriers reacted under the condition of a relatively high concentration (greater than 0.1 mM) of the sulfo-NHS material 1020, and FIG. 12B illustrates the measured barrier stability for barriers reacted under the condition of a relatively low concentration (less than 0.1 mM) of the sulfo-NHS material 1020. FIGS. 12A and 12B also include measured barrier stability for non-modified barriers which included NH₂ terminated polymer which was not reacted with the sulfo-NHS material. From FIG. 12A it may be understood that the barriers reacted under the high concentration of the sulfo-NHS material 1020 had similar stability as did the non-modified barriers. In comparison, from FIG. 12B it may be understood that the barriers reacted under the low concentration of the sulfo-NHS material 1020 had significantly higher stability than the non-modified barriers, particularly as voltage increased. FIGS. 13A and 13B schematically illustrate example reaction products for the barriers reacted as described with reference to FIGS. 12A and 12B. From the data illustrated in FIG. 12A, it may be understood that the products of the reaction using the high concentration of the sulfo-NHS material included a relatively large proportion of reaction product 1020′, as illustrated in FIG. 13A. From the data illustrated in FIG. 12B, it may be understood that the products of the reaction using the low concentration of the sulfo-NHS material included a relatively large proportion of reaction product 1020″, as illustrated in FIG. 13B.

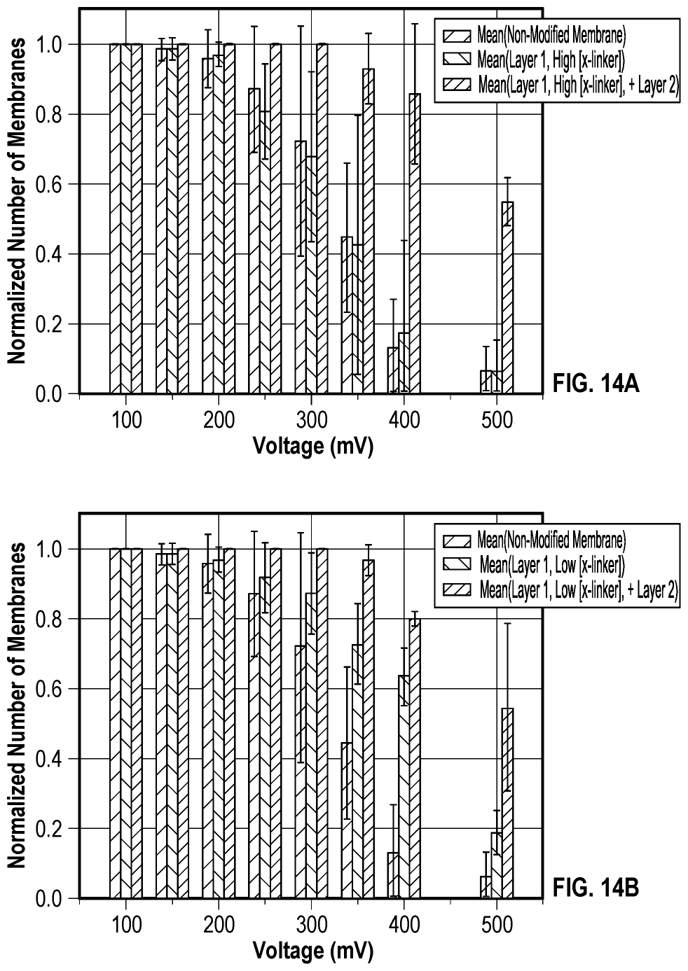

FIGS. 14A and 14B are plots of the measured barrier stability for the barriers of FIGS. 12A and 12B subsequently reacted with additional molecules. More specifically, barriers which were prepared and reacted in a manner such as described with reference to FIGS. 10, 12A-12B, and 13A-13B then were reacted with the following NH₂ compound (Mn=2 kDa, n approximately 9-10) to add another layer of molecules.

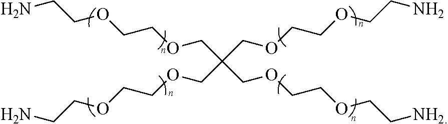

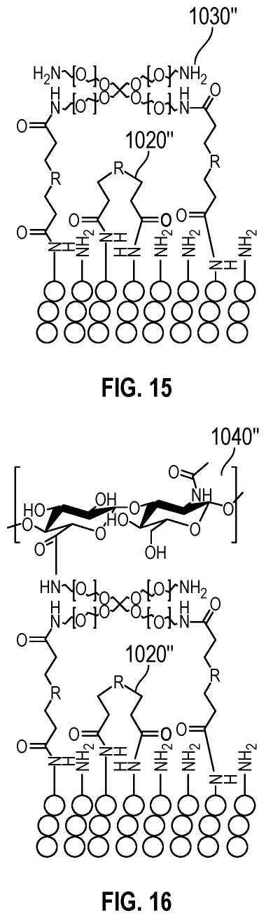

More specifically, the NH₂ compound was used in a final concentration of about 1 mg/mL solution in 1 M KCl buffer+50 mM HEPES buffer. The reaction time was about 30 minutes. After that, the reaction product of the NH₂ compound was washed with 1 M KCl buffer+50 mM HEPES buffer. From FIG. 14A it may be understood that the barriers which were initially reacted under the high concentration of the sulfo-NHS material 1020, and then reacted with the NH₂ compound above, had significantly higher stability than the non-modified barriers and than the barriers which were reacted only with the sulfo-NHS material. From FIG. 14B it may be understood that the barriers which were initially reacted under the high concentration of the sulfo-NHS material 1020, and then reacted with the NH₂ compound above, also had significantly higher stability than the non-modified barriers and than the barriers which were reacted only with the sulfo-NHS material. FIG. 15 schematically illustrates the example cross-linked reaction products 1030″ (from reaction with reaction product 1020′) as well as earlier reaction product 1020″ for the barriers reacted as described with reference to FIGS. 14A and 14B. From FIGS. 14A-14B, it may be understood that the reaction with the NH₂ compound added a layer of cross-linking reaction product 1030″ that significantly stabilized the barrier relative to barriers stabilized only using reaction products 1020′ and/or 1020″ such as described with reference to FIGS. 12A-12B and 13A-13B.

FIG. 17 is a plot of the measured barrier stability for the barrier of FIG. 14A subsequently reacted with additional molecules. More specifically, barriers which were prepared and reacted in a manner such as described with reference to FIG. 14A then were reacted with the following NHS compound (M_{w}=approximately 10 kDa, n approximately 19 or 20) to add another layer of molecules:

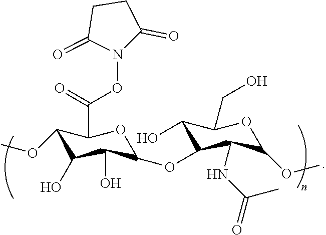

The NHS compound was used with a final concentration of about 2 mg/mL solution in 1 M KCl+50 mM HEPES buffer. The reaction time was about 30 minutes. After that the reaction product of the NHS compound was washed with 1 M KCl+50 mM HEPES buffer.

From FIG. 17 it may be understood that the barriers which were initially reacted under the high concentration of the sulfo-NHS material 1020, then reacted with the NH₂ compound above, and then reacted with the NHS compound above, had significantly higher stability than the non-modified barriers, than the barriers which were reacted only with the sulfo-NHS material, and than the barriers which were reacted with the sulfo-NHS material and then the NH₂ compound above. FIG. 16 schematically illustrates the example cross-linked reaction products 1040″ (from reaction with reaction product 1030″) as well as earlier reaction product 1020″ for the barriers reacted as described with reference to FIG. 17. From FIG. 17, it may be understood that the reaction with the NHS compound added a layer of cross-linking reaction product 1040″ that significantly stabilized the barrier relative to barriers stabilized only using reaction products 1020′ and/or 1020″ such as described with reference to FIGS. 12A-12B and 13A-13B.

### Example 2

To assess barrier stability improvement provided by reactions with molecules, barrier resistance to breakdown voltage was evaluated using an experimental setup such as described with reference to FIG. 10. In this example, the functionalized ABA triblock copolymer material “ABA1”

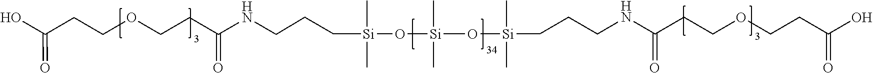

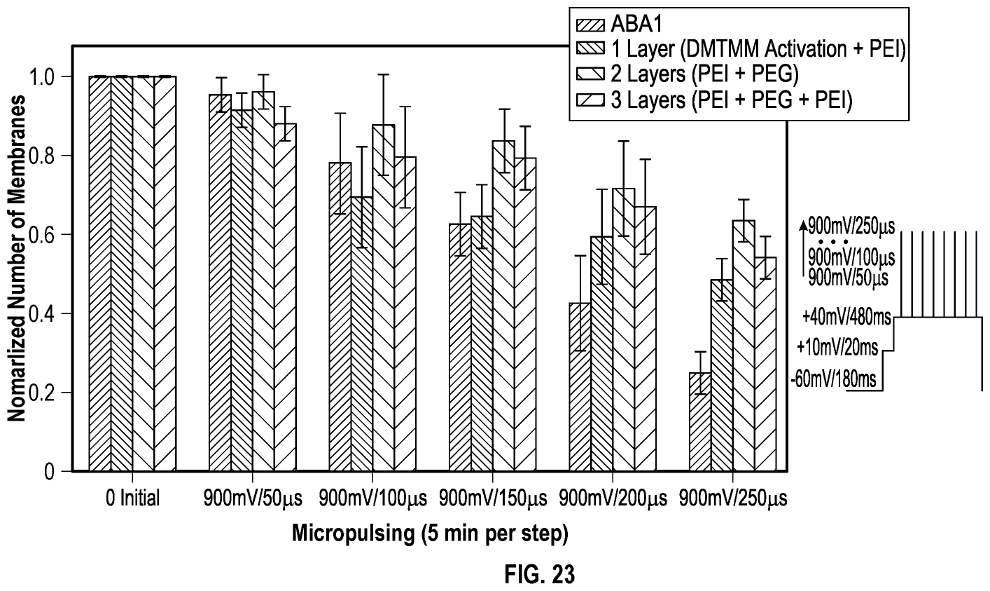

was used as the basis for the barriers. More specifically, a first set of barriers including one layer was formed by activating the carboxyl groups of ABA1 with DMTMM and reacting the activated carboxyl group with PEI, in which n=1; these barriers are referred to in FIGS. 23-25 as “1 Layer (DMTMM Activation+PEI)”. A second set of barriers including two layers was formed by reacting the first set of barriers with 4-arm PEG-NETS having a M_{w} of approximately 10 kDa, in which n=approximately 56 for each of the arms; these barriers are referred to in FIGS. 23-25 as “2 Layers (PEI+PEG).” A third set of barriers including three layers was formed by reacting the second set of barriers with PEI, in which n=1; these barriers are referred to in FIGS. 23-25 as “3 Layers (PEI+PEG+PEI).”

FIG. 23 includes a plot of the measured stability of barriers formed using ABA1 without further reaction, as well as of the first, second, and third sets of barriers described above. FIG. 23 also illustrates the voltage breakdown waveform used to assess barrier stability for this plot. A voltage of −60 mV was applied across the barrier for 180 ms, followed by a voltage of +10 mV for 20 ms, followed by a voltage of +40 mV for 480 ms. During the period of +40 mV, a series of voltage spikes of +900 mV were applied that each lasted 50 μs, divided over the next 250 μs. Between voltage spikes, the voltage was returned to +40 mV. Barrier stability was quantified as the percentage of barriers remaining at the end of each 5 minute cycle of the waveform. From FIG. 23 it may be understood that the barriers with at least one layer had higher stability than the non-modified barriers (ABA1), particularly as the pulse duration increased.

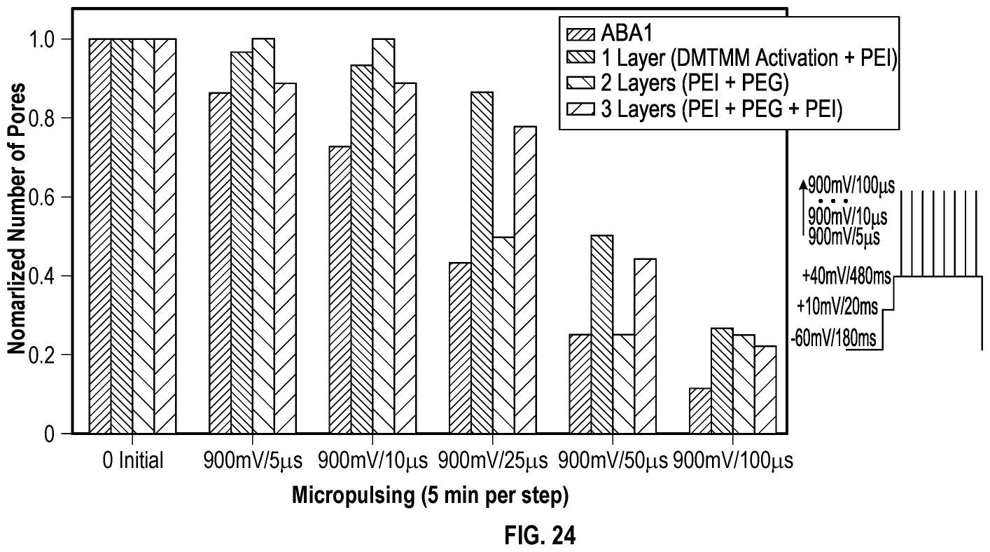

FIG. 24 is a plot of the measured stability for the barriers of FIG. 23, having MspA nanopores inserted therein. The pores were added before starting the reactions to add layers(s), and the pores were still functional after layer(s) addition. For example, the layer(s) did not affect the pore, and the pore still formed an open channel through the barrier after layer(s) addition. FIG. 24 also illustrates the voltage breakdown waveform used to assess barrier stability for this plot. A voltage of −60 mV was applied across the barrier for 180 ms, followed by a voltage of +10 mV for 20 ms, followed by a voltage of +40 mV for 480 ms. During the period of +40 mV, a series of voltage spikes of +900 mV were applied that each lasted 50 μLs, divided over the next 250 μLs. Between voltage spikes, the voltage was returned to +40 mV. Barrier stability was quantified as the percentage of barriers remaining at the end of each 5 minute cycle of the waveform. From FIG. 24, it may be understood that the barriers with at least one layer had significantly higher stability than the non-modified barriers (ABA1), particularly as the pulse duration increased.

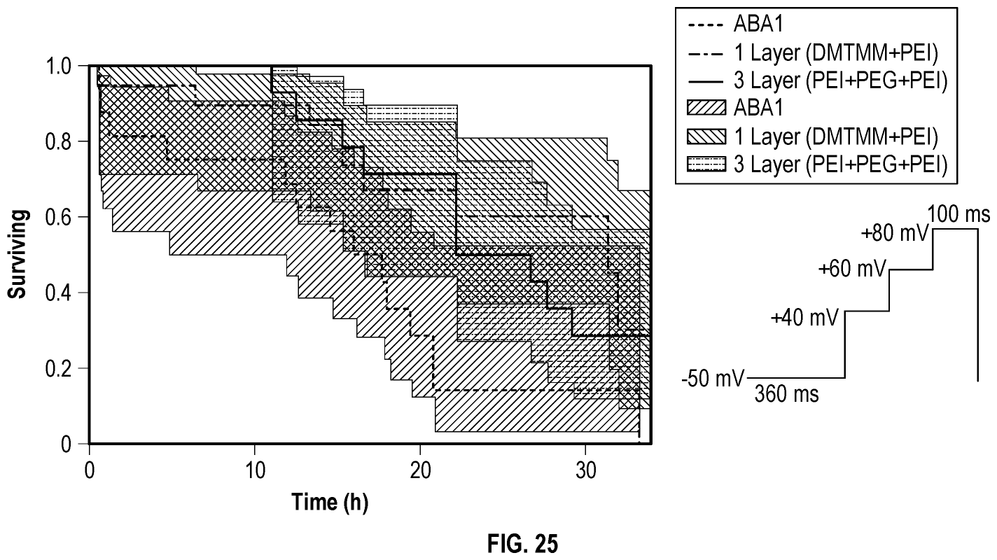

FIG. 25 includes a plot of the measured stability of the one-layer and three-layer barriers of FIG. 23, having MspA nanopores inserted therein. FIG. 25 also illustrates the voltage breakdown waveform used to assess barrier stability for this plot. Here, a voltage of −50 mV was applied across the barrier for 360 ms, followed by a voltage of +40 mV for 100 ms, followed by a voltage of +60 mV for 100 ms, followed by a voltage of +80 mV for 100 ms. Barrier stability was quantified as the normalized number of “surviving” barriers remaining at the end of repeated cycles of the waveform over the course of more than 34 hours. From FIG. 25 it may be understood that the barriers with at least one layer had significantly higher stability than the non-modified barriers. Additionally, because pores were inserted into the membranes, it may be understood that with at least one added layer there was a longer pore survival over the course of this long-lasting procedure.

### ADDITIONAL COMMENTS

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A barrier between first and second fluids, the barrier comprising:
one or more layers comprising a plurality of amphiphilic molecules; and
a first layer comprising a plurality of molecules covalently bonded to amphiphilic molecules of the plurality of amphiphilic molecules.

2. The barrier of claim 1, wherein the one or more layers comprise:
a layer comprising a first plurality of the amphiphilic molecules; and
another layer comprising a second plurality of the amphiphilic molecules contacting the first plurality of amphiphilic molecules.

3. The barrier of claim 2, wherein the molecules of the first layer are covalently bonded to amphiphilic molecules of the first plurality of amphiphilic molecules.

4. The barrier of claim 1, wherein the first layer forms an outer surface of the barrier contacting the first fluid.

5. The barrier of claim 1, further comprising a second layer comprising a plurality of molecules covalently coupled to molecules of the first layer.

6. The barrier of claim 5, wherein the second layer forms an outer surface of the barrier contacting the first fluid.

7. The barrier of claim 1, wherein at least some molecules of the first layer respectively couple to two or more molecules of the one or more layers comprising amphiphilic molecules.

8. The barrier of claim 1, wherein at least some molecules of the first layer respectively couple to only one molecule of the one or more layers comprising amphiphilic molecules.

9. The barrier of claim 1, wherein hydrophilic groups of the one or more layers of amphiphilic molecules form an outer surface of the barrier contacting the second fluid.

10. The barrier of claim 1, wherein the amphiphilic molecules comprise molecules of a diblock copolymer, the molecules of the diblock copolymer respectively comprising a hydrophobic block coupled to a hydrophilic block.

11. The barrier of claim 10, the molecules of the first layer being coupled to the hydrophilic blocks of the diblock copolymer, and
the hydrophobic blocks of the first and second pluralities of amphiphilic molecules contacting one another within the barrier.

12. The barrier of claim 1, wherein the amphiphilic molecules comprise molecules of a triblock copolymer.

13. The barrier of claim 12, each molecule of the triblock copolymer comprising first and second hydrophobic blocks and a hydrophilic block coupled to and between the first and second hydrophobic blocks.

14. The barrier of claim 13, the molecules of the first layer being coupled to the hydrophilic blocks of the triblock copolymer, and
the hydrophobic blocks of the first and second pluralities of amphiphilic molecules contacting one another within the barrier.

15. The barrier of claim 12, each molecule of the triblock copolymer comprising first and second hydrophilic blocks and a hydrophobic block coupled to and between the first and second hydrophilic blocks.

16. The barrier of claim 15, the molecules of the first layer being coupled to hydrophilic blocks of the triblock copolymer, and
the hydrophobic blocks of the first and second pluralities of amphiphilic molecules contacting one another within the barrier.

17. The barrier of claim 1, further comprising a nanopore within the barrier.

18. The barrier of claim 17, wherein the nanopore comprises α-hemolysin.

19. The barrier of claim 17, wherein the nanopore comprises MspA.

20. The barrier of claim 1, the barrier being suspended by a barrier support defining an aperture, the one or more layers being suspended across the aperture.

21. A barrier between first and second fluids, the barrier comprising:
a first layer comprising a first plurality of amphiphilic molecules, wherein the amphiphilic molecules of the first plurality of amphiphilic molecules comprise first reactive moieties.

22-38. (canceled)

39. A method of forming a barrier between first and second fluids, the method comprising:
forming one or more layers comprising a plurality of amphiphilic molecules, wherein the amphiphilic molecules comprise first reactive moieties; and
using the first reactive moieties to covalently bond a first plurality of molecules to amphiphilic molecules of the plurality of amphiphilic molecules.

40-71. (canceled)
